(19) United States
(12) Patent Application Publication
(10) Pub. No.: US 2006/0148772 A1
(43) Pub. Date: Jul. 6, 2006

(54) Combination

(76) Inventors: Astrid Hauge Evans, London (GB); Mitch Dowsett, Kent (GB); Lesley-Ann Martin, Sussex (GB); Dean Brent Evans, Oberwil (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/265,354

(22) Filed: Nov. 2, 2005

Related U.S. Application Data
(60) Provisional application No. 60/628,345, filed on Nov. 16, 2004.

(51) Int. Cl. A61K 31/57; A61K 31/56; A61K 31/496

(52) U.S. Cl. 514/171; 514/253.04; 514/396

(57) ABSTRACT

The present invention provides a combination for the treatment of a disease or condition which responds to dual EGFR and VEGF protein tyrosine kinase inhibition and either aromatase inhibition or inhibition of estrogen action, in particular a proliferative disease, especially a malignant disease, such as breast cancer, comprising a dual EGFR and VEGF protein tyrosine kinase inhibitor and either an aromatase inhibitor or an estrogen receptor antagonist for simultaneous, concurrent, separate or sequential use in reducing cell proliferation in estrogen receptor positive tumors. Also provided is a method of treating a patient suffering from a disease or condition which responds to dual EGFR and VEGF protein tyrosine kinase inhibition and either aromatase inhibition or inhibition of estrogen action comprising administering to the patient an effective amount of a dual EGFR and VEGF protein tyrosine kinase inhibitor and an effective amount of either an aromatase inhibitor or an estrogen receptor antagonist.

The present invention provides a combination for the treatment of a disease or condition which responds to dual epidermal growth factor receptor (EGFR) and vascular endothelial growth factor receptor (VEGFR) (if EGFR term is used then VEGFR term has to be used throughout the document) protein tyrosine kinase inhibition and either aromatase inhibition or inhibition of estrogen action, in particular a proliferative disease, especially a malignant disease, such as breast cancer, comprising a dual EGFR and VEGF protein tyrosine kinase inhibitor and either an aromatase inhibitor or an estrogen receptor antagonist for simultaneous, concurrent, separate or sequential use in reducing cell proliferation in hormone receptor positive tumors. Also provided is a method of treating a patient suffering from a disease or condition which responds to dual EGFR and VEGF protein tyrosine kinase inhibition and either aromatase inhibition or inhibition of estrogen action comprising administering to the patient an effective amount of a dual EGFR and VEGF protein tyrosine kinase inhibitor and an effective amount of either an aromatase inhibitor or an estrogen receptor antagonist.

### BACKGROUND OF THE INVENTION

Breast cancer continues to recur indefinitely after diagnosis in loco-regional and distant sites despite the benefits of initial surgery, radiation and medical therapies. Protein tyrosine kinase inhibitors are widely-used to inhibit protein tyrosine kinase activity in a variety of both benign and malignant diseases. Aromatase inhibitors have well-known valuable pharmacological properties. They are useful for the inhibition of estrogen biosynthesis in mammals and the treatment or prevention of estrogen dependent disorders responsive thereto, such as mammary tumours (breast carcinoma), endometriosis, premature labor and endometrial tumors in females, as well as gynecomastia in males. Estrogen receptor antagonists are useful in the treatment of breast cancer.

### SUMMARY OF THE INVENTION

It has now been found that the administration of a protein tyrosine kinase inhibitor that is a combined inhibitor EGFR and VEGF tyrosine kinases treated with either an aromatase inhibitor, such as letrozole; or an estrogen receptor antagonist, such as tamoxifen, is useful in reducing cell proliferation in hormone receptor positive tumours.

Accordingly, the present invention provides a combination, such as a combined preparation or a pharmaceutical composition, which comprises

(a) protein tyrosine kinase inhibitor; and either

(b) an aromatase inhibitor; or

(c) an estrogen receptor antagonist,

in which the active ingredients (a) and (b) or (c) are present in each case in free form or in the form of a pharmaceutically acceptable salt, for simultaneous, concurrent, separate or sequential use in the treatment of a disease or condition which responds to dual EGFR and VEGF protein tyrosine kinase inhibition and either aromatase inhibition or inhibition of estrogen action, in particular a proliferative disease, especially a malignant disease such as breast cancer.

The term “a combined preparation” defines especially a “kit of parts” in the sense that the combination partners (a) and (b) or (c) as defined above can be dosed independently or by use of different fixed combinations with distinguished amounts of the combination partners (a) and (b) or (c), i.e., simultaneously, concurrently, separately or sequentially. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) or (c) to be administered in the combined preparation can be varied, e.g., in order to cope with the needs of a patient subpopulation to be treated or the needs of the single patient which different needs can be due to the particular disease, severity of the disease, age, sex, body weight, etc. of the patients.

Further the invention provides the use of a dual EGFR and VEGF protein tyrosine kinase inhibitor, for use in combination with either an aromatase inhibitor or estrogen receptor antagonist, for treatment of a disease or condition which responds to dual EGFR and VEGF protein tyrosine kinase inhibition and either aromatase inhibition or inhibition of estrogen action, in particular a proliferative disease, especially a malignant disease, such as breast cancer.

In the alternative the invention provides the use of either an aromatase inhibitor or an estrogen receptor antagonist for the preparation of a medicament, for use in combination with a dual EGFR and VEGF protein tyrosine kinase inhibitor for treatment of a disease or condition which responds to dual EGFR and VEGF protein tyrosine kinase inhibition and either aromatase inhibition or inhibition of estrogen action, in particular a proliferative disease, especially a malignant disease, such as breast cancer.

In a further aspect, the invention provides a method of treating a patient suffering from a disease or condition which responds to dual EGFR and VEGF protein tyrosine kinase inhibition and either aromatase inhibition or inhibition of estrogen action, in particular, a proliferative disease, especially a malignant disease, such as breast cancer, comprising administering to the patient an effective amount of a dual EGFR and VEGF protein tyrosine kinase inhibitor and an effective amount of either an aromatase inhibitor or an estrogen receptor antagonist.

In yet further aspects the invention provides:
(i) A package comprising a dual EGFR and VEGF protein tyrosine kinase inhibitor together with instructions for use in combination with either an aromatase inhibitor or an estrogen receptor antagonist for treatment of a disease or condition which responds to dual EGFR and VEGF protein tyrosine kinase inhibition and either aromatase inhibition or inhibition of estrogen action, in particular, a proliferative disease, especially malignant disease, such as breast cancer; or
(ii) a package comprising either an aromatase inhibitor or an estrogen receptor antagonist together with instructions for use in combination with a dual EGFR and VEGF protein tyrosine kinase inhibitor for treatment of a disease or condition which responds to dual EGFR and VEGF protein tyrosine kinase inhibition and either aromatase inhibition or inhibition of estrogen action, in particular a proliferative disease, especially malignant disease, such as breast cancer.

Diseases and conditions which may be treated in accordance with the present invention include any of those diseases/conditions which may be treated using a dual EGFR and VEGF protein tyrosine kinase inhibitor, i.e., “a disease or condition which responds to the inhibition of the tyrosine kinase activity of the EGFR or VEGF families” and either any diseases/conditions which may be treated using an aromatase inhibitor, i.e., “a disease or condition which responds to aromatase inhibition”, or any diseases/conditions which may be treated using an estrogen receptor antagonist, i.e., “a disease or condition which responds to the inhibition of estrogen action”. A combination of the invention comprising the dual EGFR and VEGF protein tyrosine kinase inhibitor of the present invention and an aromatase inhibitor such as, but not limited to, letrozole, is particularly useful for the treatment of hormone receptor positive tumors, e.g., breast tumors. A combination of the invention comprising the dual EGFR and VEGF protein tyrosine kinase inhibitor of the present invention and an estrogen receptor antagonist, such as but not limited to, tamoxifen, is particularly useful for the treatment of estrogen receptor positive tumors, e.g., breast tumors.

In a particularly preferred embodiment, the invention provides a method of reducing cell proliferation in hormone receptor positive breast tumours comprising administering an effective amount of a dual EGFR and VEGF protein tyrosine kinase inhibitor and an effective amount of either an aromatase inhibitor, such as but not limited to, letrozole, or an effective amount of an estrogen receptor antagonist, such as but not limited to, tamoxifen.

Thus, in the present description the terms “treatment” or “treat” refer to both prophylactic or preventative treatment, as well as curative or disease modifying treatment, including treatment of patients at risk of contracting the disease or suspected to have contracted the disease, as well as patient who are ill or have been diagnosed as suffering from a disease or medical condition.

### DETAILED DESCRIPTION OF THE DRAWINGS

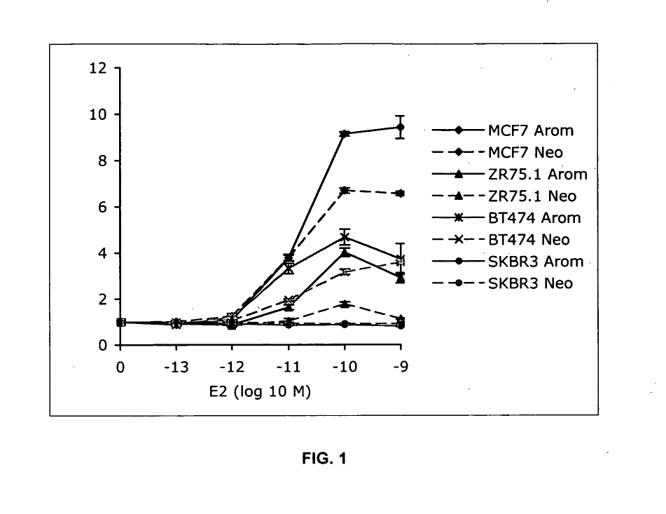

FIG. 1 illustrates the responsiveness of Aromatase Expressing Clones to E2.

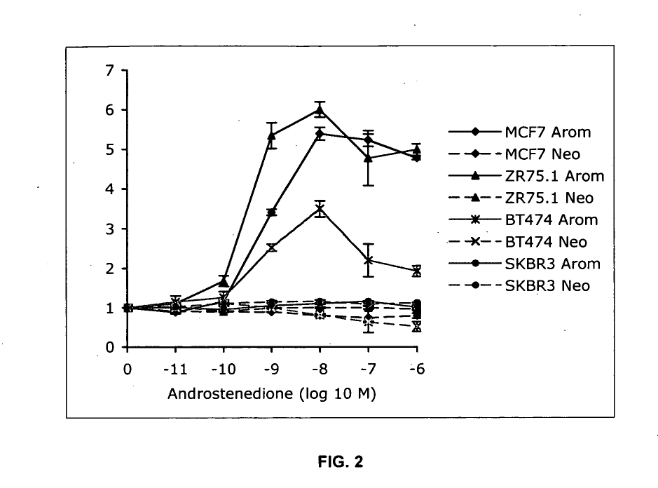

FIG. 2 illustrates the proliferative response of Aromatase Expressing Clones to androstenedione.

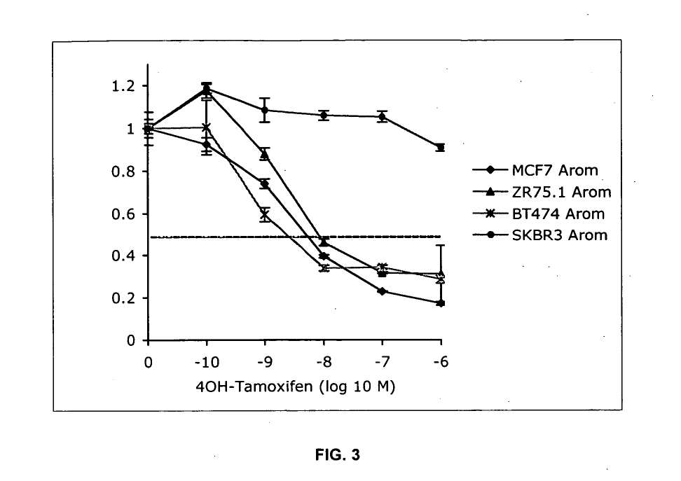

FIG. 3 illustrates the proliferative response of Aromatase Expressing Clones to 4-OH tamoxifen.

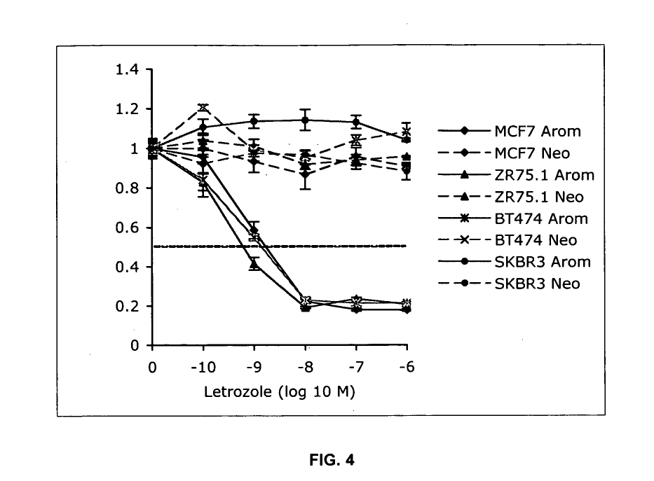

FIG. 4 illustrates the proliferative response of Aromatase Expressing Clones to Letrozole.

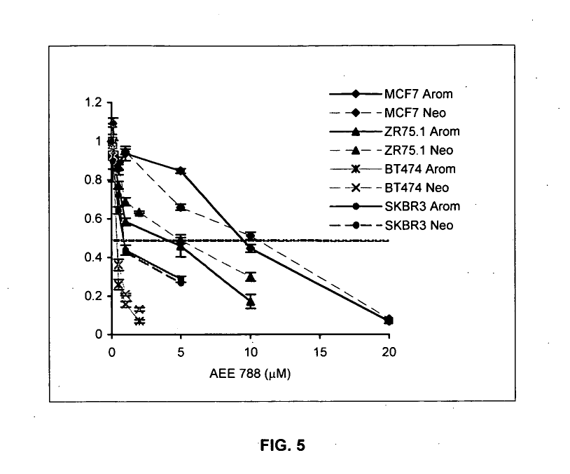

FIG. 5 illustrates the proliferation of aromatase and neo expressing clones in response to 6-[4-(4-ethyl-piperazin-1-ylmethyl)-phenyl]-7H-pyrrolo[2,3-d]pyrimidin-4-yl}-(1-[phenyl-ethyl)-amine.

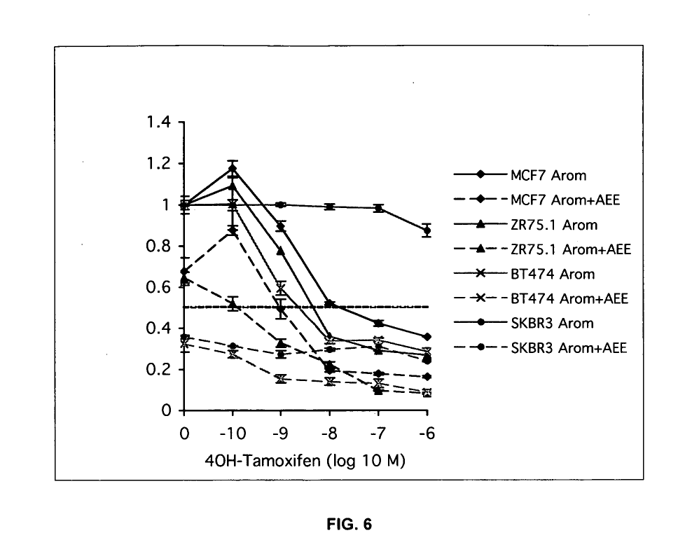

FIG. 6 illustrates the proliferation of aromatase expressing clones in response to 6-[4-(4-ethyl-piperazin-1-ylmethyl)-phenyl]-7H-pyrrolo[2,3-d]pyrimidin-4-yl}-(1-[phenyl-ethyl)-amine in combination with 4-OH tamoxifen.

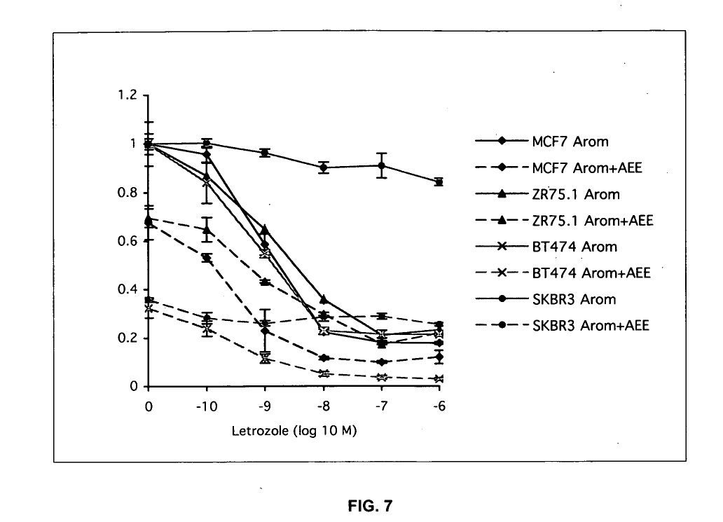

FIG. 7 illustrates the proliferation of aromatase expressing clones in response to 6-[4-(4-ethyl-piperazin-1-ylmethyl)-phenyl]-7H-pyrrolo[2,3-d]pyrimidin-4-yl}-(1-[phenyl-ethyl)-amine in combination with letrozole.

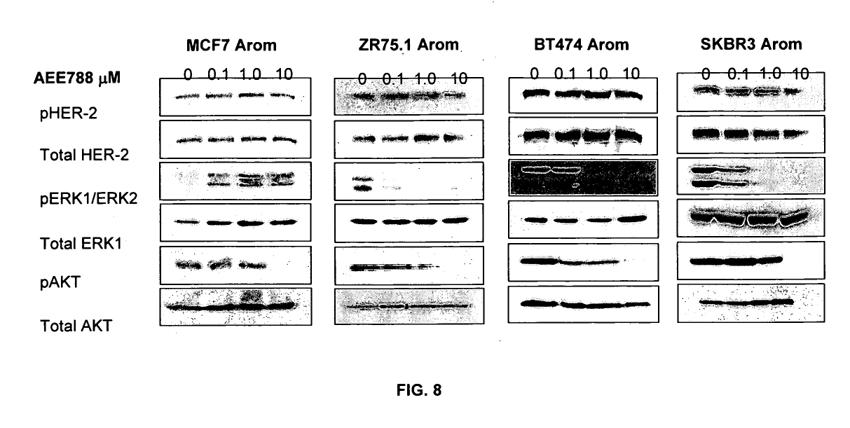

FIG. 8 illustrates a Western blot showing the effect of increasing doses of AEE788 on HER2, MAPK and AKT signal transduction.

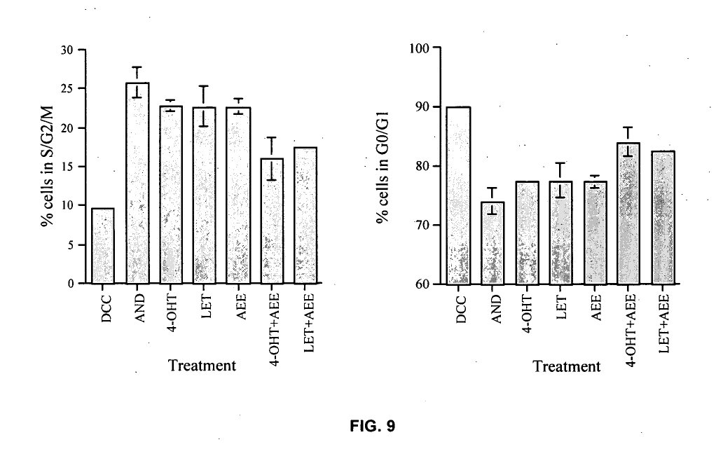

FIG. 9 illustrates the effect of 4-OH tamoxifen or letrozole±AEE788 on cell cycle arrest in MCF72A cells.

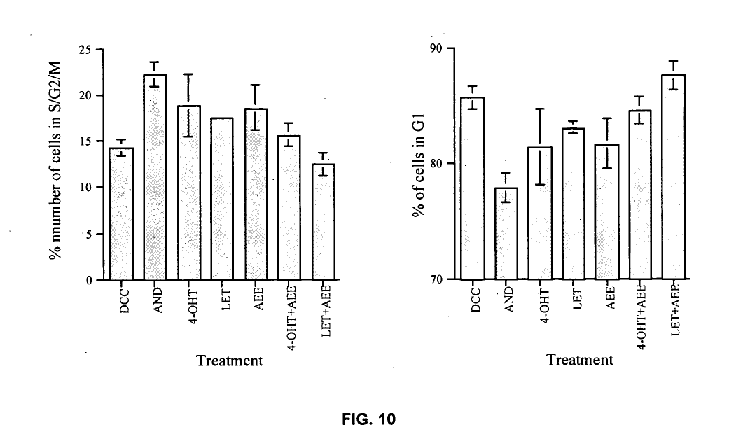

FIG. 10 illustrates the effect of 4-OH tamoxifen or letrozole±AEE788 on cell cycle arrest in ZR75.1 A3 cells.

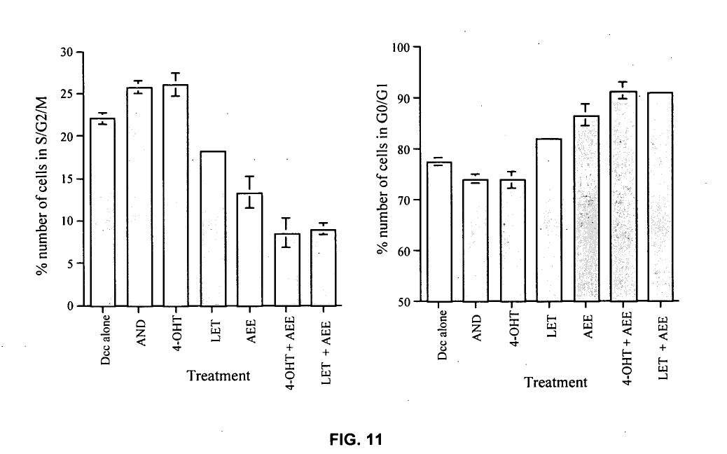

FIG. 11 illustrates the effect of 4-OH tamoxifen or letrozole±AEE788 on cell cycle arrest in BT474 A3 cells.

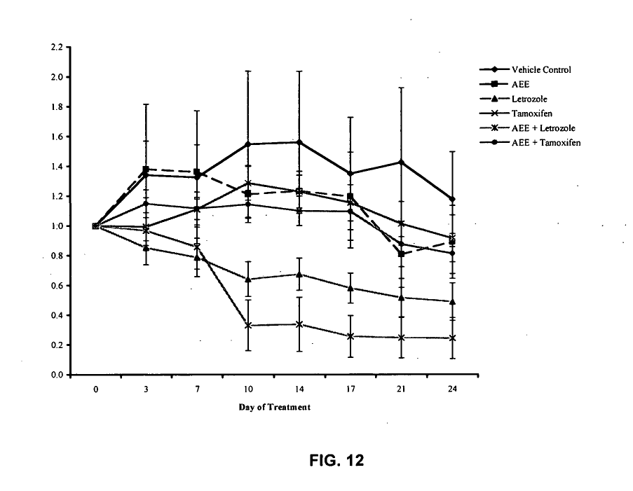

FIG. 12 illustrates the effect of letrozole, 4-OH tamoxifen or AEE788 alone or in combination on ZR75.1 A3 tumours.

### DETAILED DESCRIPTION OF THE INVENTION

The dual EGFR and VEGF protein tyrosine kinase inhibitors are described in WO 03/013541, which is incorporated by reference, and are 7H-pyrrolo[2,3-d]pyrimidine derivatives of formula (I): 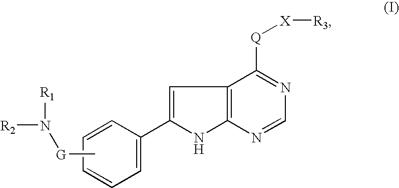
wherein
R₁ and R₂ are, each independently of the other hydrogen, unsubstituted or substituted alkyl or cycloalkyl, a heterocyclic radical bonded via a ring carbon atom, or a radical of the formula R₄—Y—(C=Z)-,
wherein
   R₄ is unsubstituted, mono- or di-substituted amino or a heterocyclic radical;
   Y is either not present or lower alkyl; and
   Z is oxygen, sulfur or imino, with the proviso that R₁ and R₂ are not both hydrogen, or
R₁ and R₂, together with the nitrogen atom to which they are attached, form a heterocyclic radical;
R₃ is a heterocyclic radical or an unsubstituted or substituted aromatic radical;
G is C₁-C₇alkylene, —C(═O)— or C₁-C₆-alkylene-C(═O)—, wherein the carbonyl group is attached to the NR₁R₂ moiety;
Q is —NH— or —O—, with the proviso that Q is —O— if G is —C(═O)— or C₁-C₆alkylene-C(═O)—; and
X is either not present or C₁-C₇alkylene, with the proviso that a heterocyclic radical R₃ is bonded via a ring carbon atom if X is not present;
   or a salt of the compounds.

The general terms used hereinbefore and hereinafter preferably have within the context of this disclosure the following meanings, unless otherwise indicated:

Where the plural form is used for compounds, salts and the like, this is taken to mean also a single compound, salt or the like.

Where compounds of formula (I) are mentioned which can form tautomers, it is meant to include also the tautomers of such compounds of formula (I). In particular, tautomerism occurs, e.g., for compounds of formula (I) which contain a 2-hydroxy-pyridyl radical. See, e.g., radical R₃ of the below-mentioned Examples 115-120. In such compounds the 2-hydroxy-pyridyl radical can also be present as pyrid-2(1H)-on-yl.

Asymmetric carbon atoms of a compound of formula (I) that are optionally present may exist in the (R), (S) or (R,S) configuration, preferably in the (R) or (S) configuration. Substituents at a double bond or a ring may be present in cis- (=Z-) or trans (=E-) form. The compounds may thus be present as mixtures of isomers or preferably as pure isomers.

Preferably alkyl contains up to 20 carbon atoms and is most preferably lower alkyl.

The prefix “lower” denotes a radical having up to and including a maximum of 7 carbon atoms, especially up to and including a maximum of 4 carbon atoms, the radicals in question being either unbranched or branched with single- or multiple-branching.

Lower alkyl is, e.g., methyl, ethyl, n-propyl, isopropyl, n-butyl, isobutyl, sec-butyl, tert-butyl, n-pentyl, isopentyl, neopentyl, n-hexyl or n-heptyl.

Alkyl R₁ and R₂, independently of each other, are preferably methyl, ethyl, isopropyl or tert-butyl, especially methyl or ethyl.

Lower alkyl Y is preferably methyl, ethyl or propyl.

Lower alkoxy is, e.g., ethoxy or methoxy, especially methoxy.

Substituted alkyl is preferably lower alkyl as defined above where one or more, preferably one substituent may be present, such as, e.g., amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio, halogen or a heterocyclic radical.

Substituted alkyl R₁ and R₂ are, independently of each other, preferably hydroxy-lower alkyl, N,N-di-lower alkylamino-lower alkyl or morpholinyl-lower alkyl.

Preferably unsubstituted or substituted cycloalkyl R₁ or R₂ contains from 3 carbon atoms up to 20 carbon atoms and is especially unsubstituted or also substituted C₃-C₆cycloalkyl wherein the substituents are selected from, e.g., unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio, halogen or a heterocyclic radical.

Mono- or di-substituted amino is amino substituted by one or two radicals selected independently of one another from, e.g., unsubstituted or substituted lower alkyl.

Disubstituted amino R₄ is preferably N,N-di-lower alkylamino, especially N,N-dimethylamino or N,N-diethylamino.

A heterocyclic radical contains especially up to 20 carbon atoms and is preferably a saturated or unsaturated monocyclic radical having from 4- or 8-ring members and from 1-3 heteroatoms which are preferably selected from nitrogen, oxygen and sulfur, or a bi- or tri-cyclic radical wherein, e.g., one or two carbocyclic radicals, such as, e.g., benzene radicals, are annellated (fused) to the mentioned monocyclic radical. If a heterocyclic radical contains a fused carbocyclic radical then the heterocyclic radical may also be attached to the rest of the molecule of formula (I) via a ring atom of the fused carbocyclic radical. The heterocyclic radical, including the fused carbocyclic radical(s) if present, is optionally substituted by one or more, preferably by one or two, radicals, such as, e.g., unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio or halogen.

Most preferably a heterocyclic radical is pyrrolidinyl, piperidyl, lower alkyl-piperazinyl, di-lower alkyl-piperazinyl, morpholinyl, tetrahydropyranyl, pyridyl, pyridyl substituted by hydroxy or lower alkoxy or benzodioxolyl, especially pyrrolidinyl, piperidyl, lower alkyl-piperazinyl, di-lower alkyl-piperazinyl or morpholinyl.

A heterocyclic radical R₁ or R₂ is as defined above for a heterocyclic radical with the proviso that it is bonded to the rest of the molecule of formula (I) via a ring carbon atom. Preferably a heterocyclic radical R₁ or R₂ is lower alkyl-piperazinyl or especially preferred tetrahydropyranyl. If one of the two radicals R₁ and R₂ represents a heterocyclic radical, the other is preferably hydrogen.

A heterocyclic radical R₃ is as defined above for a heterocyclic radical with the proviso that it is bonded to Q via a ring carbon atom if X is not present. Preferably, a heterocyclic radical R₃ is benzodioxolyl, pyridyl substituted by hydroxy or lower alkoxy, or especially preferred indolyl substituted by halogen and lower alkyl. If R₃ is pyridyl substituted by hydroxy then the hydroxy group is preferably attached to a ring carbon atom adjacent to the ring nitrogen atom.

A heterocyclic radical R₄ is as defined above for a heterocyclic radical and is preferably pyrrolidinyl, piperidyl, lower alkyl-piperazinyl, morpholinyl or pyridyl.

If R₁ and R₂, together with the nitrogen atom to which they are attached, form a heterocyclic radical, the heterocyclic radical is as defined above for a heterocyclic radical and represents preferably pyrrolidinyl, piperidyl, lower alkyl-piperazinyl, di-lower alkyl-piperazinyl or morpholinyl.

An unsubstituted or substituted aromatic radical R₃ has up to 20 carbon atoms and is unsubstituted or substituted, e.g., in each case unsubstituted or substituted phenyl.

Preferably an unsubstituted aromatic radical R₃ is phenyl. A substituted aromatic radical R₃ is preferably phenyl substituted by one or more substituents selected independently of one another from the group consisting of unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio and halogen. Most preferably a substituted aromatic radical R₃ is phenyl substituted by one or more radicals selected independently of one another from the group consisting of lower alkyl, amino, hydroxy, lower alkoxy, halogen and benzyloxy.

Halogen is primarily fluoro, chloro, bromo or iodo, especially fluoro, chloro or bromo.

C₁-C₇Alkylene may be branched or unbranched and is in particular C₁-C₃-alkylene.

C₁-C₇Alkylene G is preferably C₁-C₃alkylene, most preferably methylene (—CH₂—).

If G is not C₁-C₇alkylene it preferably represents —C(═O)—.

C₁-C₇Alkylene X is preferably C₁-C₃alkylene, most preferably methylene (—CH₂—) or ethan-1,1-diyl(—CH(CH₃)—).

Q is preferably —NH—.

Z is preferably oxygen or sulfur, most preferably oxygen.

Salts are especially the pharmaceutically acceptable salts of compounds of formula (I).

Such salts are formed, e.g., as acid addition salts, preferably with organic or inorganic acids, from compounds of formula (I) with a basic nitrogen atom, especially the pharmaceutically acceptable salts.

In the presence of negatively-charged radicals, such as carboxy or sulfo; salts may also be formed with bases, e.g., metal or ammonium salts, such as alkali metal or alkaline earth metal salts; or ammonium salts with ammonia or suitable organic amines, such as tertiary monoamines.

In the presence of a basic group and an acid group in the same molecule, a compound of formula (I) may also form internal salts.

In view of the close relationship between the novel compounds in free form and in the form of their salts, including those salts that can be used as intermediates, e.g., in the purification or identification of the novel compounds, hereinbefore and hereinafter any reference to the free compounds is to be understood as referring also to the corresponding salts, as appropriate and expedient.

In one embodiment, a particularly preferred dual EGFR and VEGF protein tyrosine kinase inhibitor for use in the invention is {6-[4-(4-ethyl-piperazin-1-ylmethyl)-phenyl]-7H-pyrrolo[2,3-d]pyrimidin-4-yl}-(1-[phenyl-ethyl)-amine or a pharmaceutically acceptable salt thereof.

Suitable aromatase inhibitors for use in the invention may include the following compounds or derivatives thereof or pharmaceutically acceptable salts thereof, or any hydrate or solvate thereof: steroidal, especially exemestane and formestane and, in particular non-steroidal, especially aminoglutethimide, vorozole, fadrozole, anastrozole and, very especially, letrozole. Further suitable aromatase inhibitors for use in the present invention are roglethimide, pyridoglutethimide, trilostane, testolactone, atamestane, 1-methyl-1,4-androstadiene-3,17-dione, ketokonazole [see also Cancer Treat Res, Vol. 94, pp. 231-254 (1998) and WO 99/30708] and YM511 [Susaki et al., J Ster Biochem Mol Biol, Vol. 58, pp. 189-194 (1996)] or derivatives thereof or pharmaceutically acceptable salts thereof, or any hydrate or solvate thereof. Preferably, an aromatase inhibitor is selected from exemestane, formestane, aminoglutethimide, fadrozole, anastrozole and letrozole. Exemestane can be administered, e.g., in the form as it is marketed, e.g., under the trademark AROMASIN™. Formestane can be administered, e.g., in the form as it is marketed, e.g., under the trademark LENTARON™. Fadrozole can be administered, e.g., in the form as it is marketed, e.g., under the trademark AFEMA™. Anastrozole can be administered, e.g., in the form as it is marketed, e.g., under the trademark ARIMIDEX™. Letrozole can be administered, e.g., in the form as it is marketed, e.g., under the trademark FEMARA™ or FEMAR™. Aminoglutethimide can be administered, e.g., in the form as it is marketed, e.g., under the trademark ORIMETEN™.

Furthermore, the structure of the active agents mentioned herein by name may be taken from the actual edition of the standard compendium “The Merck Index” or from databases, e.g., Patents International, e.g., IMS World Publications. The corresponding content thereof is hereby incorporated by reference. Any person skilled in the art is fully-enabled, based on these references, to manufacture and test the pharmaceutical indications and properties in standard test models/methods, both in vitro and in vivo.

Standard test methods to measure aromatase inhibitory activity of a compound in vitro and in vivo are well-known in the art. See, e.g, J Biol Chem, Vol. 249, p. 5364 (1974); J Enzyme Inhib, Vol. 4, 169 (1990); and J Enzyme Inhib, Vol. 4, p. 179 (1990).

The following compounds and groups of compounds listed below under (a)-(z) and (aa)-(ae) represent further aromatase inhibitors. Each individual group forms a group of aromatase inhibitors that can be used in accordance with the present invention.

(a) The compounds of formulae (I) and (I*), as defined in EP A 165 904. These are especially the compounds of formula (I) 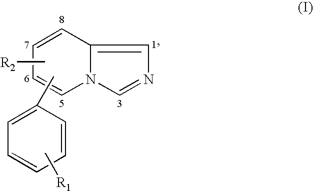
wherein
R₁ is hydrogen, lower alkyl; lower alkyl substituted by hydroxy, lower alkoxy, lower alkanoyloxy, lower alkanoyl, amino, lower alkylamino, di-lower alkylamino, halogen, sulfo, carboxy, lower alkoxycarbonyl, carbamoyl or by cyano, nitro, halogen, hydroxy, lower alkoxy, lower alkanoyloxy, phenylsulfonyloxy, lower alkylsulfonyloxy, mercapto, lower alkylthio, lower alkylsulfinyl, lower alkylsulfonyl, lower alkanoylthio, amino, lower alkylamino, di-lower alkylamino, lower alkyleneamino, N-morpholino, N-thiomorpholino, N-piperazino that is unsubstituted or lower alkyl-substituted in the 4-position, tri-lower alkylammonio, sulfo, lower alkoxysulfonyl, sulfamoyl, lower alkylsulfamoyl, di-lower alkylsulfamoyl, formyl, iminomethyl that is unsubstituted or substituted at the nitrogen atom by hydroxy, lower alkoxy, lower alkanoyloxy, lower alkyl, phenyl or by amino, C₂-C₇alkanoyl, benzoyl, carboxy, lower alkoxycarbonyl, carbamoyl, lower alkylcarbamoyl, di-lower alkylcarbamoyl, cyano, 5-tetrazolyl, unsubstituted or lower alkyl-substituted 4,5-dihydro-2-oxazolyl or hydroxycarbamoyl; and
R₂ is hydrogen, lower alkyl, phenyl-lower alkyl, carboxy-lower alkyl, lower alkoxycarbonyl-lower alkyl, halogen, hydroxy, lower alkoxy, lower alkanoyloxy, mercapto, lower alkylthio, phenyl-lower alkylthio, phenylthio, lower alkanoylthio, carboxy, lower alkoxycarbonyl or lower alkanoyl;
the 7,8-dihydro derivatives thereof; and the compounds of formula (I*) 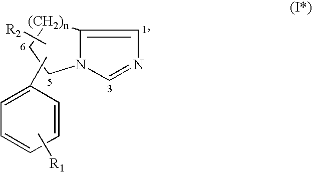
   wherein
n is 0, 1, 2, 3 or 4; and
R₁ and R₂ are as defined above for formula (I);
it being possible for the phenyl ring in the radicals phenylsulfonyloxy, phenyliminomethyl, benzoyl, phenyl-lower alkyl, phenyl-lower alkylthio and phenylthio to be unsubstituted or substituted by lower alkyl, lower alkoxy or by halogen;
it being possible in a compound of formula (I*) for the two substituents C₆H₄—R, and R₂ to be linked to each of the saturated carbon atoms of the saturated ring, either both to the same carbon atom or both to different carbon atoms;
   and pharmaceutically acceptable salts thereof.

Preferred compounds of this group are:
1. 5-(p-cyanophenyl)imidazo[1,5-a]pyridine;
2. 5-(p-ethoxycarbonylphenyl)imidazo[1,5-a]pyridine;
3. 5-(p-carboxyphenyl)imidazo[1,5-a]pyridine;
4. 5-(p-tert-butylaminocarbonylphenyl)imidazo[1,5-a]pyridine;
5. 5-(p-ethoxycarbonylphenyl)-5,6,7,8-tetrahydroimidazo[1,5-a]pyridine;
6. 5-(p-carboxyphenyl)-5,6,7,8-tetrahydroimidazo[1,5-a]pyridine;
7. 5-(p-carbamoylphenyl)-5,6,7,8-tetrahydroimidazo[1,5-a]pyridine;
8. 5-(p-tolyl)-5,6,7,8-tetrahydroimidazo[1,5-a]pyridine;
9. 5-(p-hydroxymethylphenyl)imidazo[1,5-a]pyridine;
10. 5-(p-cyanophenyl)-7,8-dihydroimidazo[1,5-a]pyridine;
11. 5-(p-bromophenyl)-5,6,7,8-tetrahydroimidazo[1,5-a]pyridine;
12. 5-(p-hydroxymethylphenyl)-5,6,7,8-tetrahydroimidazo[1,5-a]pyridine;
13. 5-(p-formylphenyl)-5,6,7,8-tetrahydroimidazo[1,5-a]pyridine;
14. 5-(p-cyanophenyl)-5-methylthio-5,6,7,8-tetrahydroimidazo[1,5-a]pyridine;
15. 5-(p-cyanophenyl)-5-ethoxycarbonyl-5,6,7,8-tetrahydroimidazo[1,5-a]pyridine;
16. 5-(p-aminophenyl)-5,6,7,8-tetrahydroimidazo[1,5-a]pyridine;
17. 5-(p-formylphenyl)imidazo[1,5-a]pyridine;
18. 5-(p-carbamoylphenyl)imidazo[1,5-a]pyridine;
19. 5H-5-(4-tert-butylaminocarbonylphenyl)-6,7-dihydropyrrolo[1,2-c]imidazole;
20. 5H-5-(4-cyanophenyl)-6,7-dihydropyrrolo[1,2-c]imidazole;
21. 5H-5-(4-cyanophenyl)-6,7,8,9-tetrahydroimidazo[1,5-a]azepine;
22. 5-(4-cyanophenyl)-6-ethoxycarbonylmethyl-5,6,7,8-tetrahydroimidazo[1,5-a]pyridine;
23. 5-(4-cyanophenyl)-6-carboxymethyl-5,6,7,8-tetrahydroimidazo[1,5-a]pyridine;
24. 5-benzyl-5-(4-cyanophenyl)-5,6,7,8-tetrahydroimidazo[1,5-a]pyridine;
25. 7-(p-cyanophenyl)-5,6,7,8-tetrahydroimidazo[1,5-a]pyridine;
26. 7-(p-carbamoylphenyl)-5,6,7,8-tetrahydroimidazo[1,5-a]pyridine; and
27. 5-(p-cyanophenyl)-5,6,7,8-tetrahydroimidazo[1,5-a]pyridine (=fadrozole).

(b) The compounds of formula (I), as defined in EP A 236 940. These are especially the compounds of formula (I) 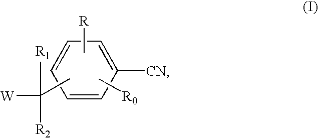
wherein
R and R₀, independently of one another, are each hydrogen or lower alkyl, or
R and R₀ at adjacent carbon atoms, together with the benzene ring to which they are bonded, form a naphthalene or tetrahydronaphthalene ring;
R₁ is hydrogen, lower alkyl, aryl, aryl-lower alkyl or lower alkenyl;
R₂ is hydrogen, lower alkyl, aryl, aryl-lower alkyl, (lower alkyl, aryl or aryl-lower alkyl)-thio or lower alkenyl, or
R₁ and R₂, together, are lower alkylidene or C₄-C₆alkylene;
W is 1-imidazolyl, 1-(1,2,4 or 1,3,4)-triazolyl, 3-pyridyl or one of the mentioned heterocyclic radicals substituted by lower alkyl, and aryl within the context of the above definitions has the following meanings: phenyl that is unsubstituted or substituted by one or two substituents from the group lower alkyl, lower alkoxy, hydroxy, lower alkanoyloxy, nitro, amino, halogen, trifluoromethyl, cyano, carboxy, lower alkoxycarbonyl, carbamoyl, N-lower alkylcarbamoyl, N,N-di-lower alkylcarbamoyl, lower alkanoyl, benzoyl, lower alkylsulfonyl, sulfamoyl, N-lower alkylsulfamoyl and N,N-di-lower alkylsulfamoyl; also thienyl, indolyl, pyridyl or furyl, or one of the four last-mentioned heterocyclic radicals monosubstituted by lower alkyl, lower alkoxy, cyano or by halogen;
   and pharmaceutically acceptable salts thereof.

Preferred compounds of this group are:
1. 4-[α-(4-cyanophenyl)-1-imidazolylmethyl]-benzonitrile;
2. 4-[α-(3-pyridyl)-1-imidazolylmethyl]-benzonitrile;
3. 4-[α-(4-cyanobenzyl)-1-imidazolylmethyl]-benzonitrile;
4. 1-(4-cyanophenyl)-1-(1-imidazolyl)-ethylene;
5. 4-[α-(4-cyanophenyl)-1-(1,2,4-triazolyl)methyl]-benzonitrile (=letrozole); and
6. 4-[α-(4-cyanophenyl)-3-pyridylmethyl]-benzonitrile.

(c) The compounds of formula (I), as defined in EP A 408 509. These are especially the compounds of formula (I) 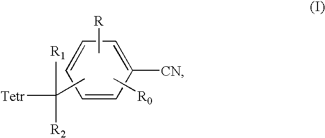
wherein
Tetr is 1- or 2-tetrazolyl that is unsubstituted or substituted in the 5-position by lower alkyl, phenyl-lower alkyl or by lower alkanoyl;
R₁ and R₂, independently of one another, are each hydrogen, lower alkyl that is unsubstituted or substituted by hydroxy, lower alkoxy, halogen, carboxy, lower alkoxycarbonyl, (amino, lower alkylamino or di-lower alkylamino)-carbonyl or by cyano, lower alkenyl, aryl, heteroaryl, aryl-lower alkyl, C₃-C₆cycloalkyl, C₃-C₆cycloalkyl-lower alkyl, lower alkylthio, arylthio or aryl-lower alkylthio, or
R₁ and R₂, together, are straight-chained C₄-C₆alkylene that is unsubstituted or substituted by lower alkyl, or are a group —(CH₂)ₘ-1,2-phenylene-(CH₂)ₙ—, wherein m and n, independently of one another, are each 1 or 2 and 1,2-phenylene is unsubstituted or substituted in the same way as phenyl in the definition of aryl below, or are lower alkylidene that is unsubstituted or mono- or di-substituted by aryl; and
R and R₀, independently of one another, are each hydrogen or lower alkyl, or
R and R₀ together, located at adjacent carbon atoms of the benzene ring, are a benzo group that is unsubstituted or substituted in the same way as phenyl in the definition of aryl below; aryl in the above definitions being phenyl that is unsubstituted or substituted by one or more substituents from the group consisting of lower alkyl, lower alkoxy, hydroxy, lower alkanoyloxy, nitro, amino, halogen, trifluoromethyl, carboxy, lower alkoxycarbonyl, (amino, lower alkylamino or di-lower alkylamino)-carbonyl, cyano, lower alkanoyl, benzoyl, lower alkylsulfonyl and (amino, lower alkylamino or di-lower alkylamino)-sulfonyl, heteroaryl in the above definitions being an aromatic heterocyclic radical from the group consisting of pyrrolyl, pyrazolyl, imidazolyl, triazolyl, tetrazolyl, furanyl, thienyl, isoxazolyl, oxazolyl, oxadiazolyl, isothiazolyl, thiazolyl, thiadiazolyl, pyridyl, pyridazinyl, pyrimidyl, pyrazinyl, triazinyl, indolyl, isoindolyl, benzimidazolyl, benzotriazolyl, benzofuranyl, benzothienyl, benzoxazolyl, benzothiazolyl, benzoxadiazolyl, benzothiadiazolyl, quinolyl and isoquinolyl that is unsubstituted or substituted in the same way as phenyl in the definition of aryl above;
   and pharmaceutically acceptable salts thereof.

Preferred compounds of this group are:
1. 4-(2-tetrazolyl)methyl-benzonitrile;
2. 4-[α-(4-cyanophenyl)-(2-tetrazolyl)methyl]-benzonitrile;
3. 1-cyano-4-(1-tetrazolyl)methyl-naphthalene; and
4. 4-[α-(4-cyanophenyl)-(1-tetrazolyl)methyl]-benzonitrile.

(d) The compounds of formula (I), as defined in EP 91810110.6. These are especially the compounds of formula (I) 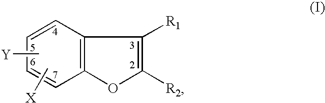
wherein
X is halogen, cyano, carbamoyl, N-lower alkylcarbamoyl, N-cycloalkyl-lower alkylcarbamoyl, N,N-di-lower alkylcarbamoyl, N-arylcarbamoyl, hydroxy, lower alkoxy, aryl-lower alkoxy or aryloxy, wherein aryl is phenyl or naphthyl, each of which is unsubstituted or substituted by lower alkyl, hydroxy, lower alkoxy, halogen and/or by trifluoromethyl;
Y is a group —CH₂-A, wherein A is 1-imidazolyl, 1-(1,2,4-triazolyl), 1-(1,3,4-triazolyl), 1-(1,2,3-triazolyl), 1-(1,2,5-triazolyl), 1-tetrazolyl or 2-tetrazolyl, or
Y is hydrogen;
R₁ and R₂, independently of one another, are each hydrogen, lower alkyl or a group —CH₂-A as defined for Y, or
R₁ and R₂, together, are —(CH₂)ₙ—, wherein n is 3, 4 or 5, with the proviso that one of the radicals Y, R₁ and R₂ is a group —CH₂-A, with the further proviso that in a group —CH₂-A as a meaning of R₁ or R₂, A is other than 1-imidazolyl when X is bromine, cyano or carbamoyl, and with the proviso that in a group —CH₂-A as a meaning of Y, A is other than 1-imidazolyl when X is halogen or lower alkoxy;
R₁ is hydrogen; and
R₂ is hydrogen or lower alkyl;
   and pharmaceutically acceptable salts thereof.

Preferred compounds of this group are:
1. 7-cyano-4-[1-(1,2,4-triazolyl)methyl]-2,3-dimethylbenzofuran;
2. 7-cyano-4-(1-imidazolylmethyl)-2,3-dimethylbenzofuran;
3. 7-carbamoyl-4-(1-imidazolylmethyl)-2,3-dimethylbenzofuran; and
4. 7-N-(cyclohexylmethyl)carbamoyl-4-(1-imidazolylmethyl)-2,3-dimethylbenzofuran.

(e) The compounds of formula (I), as defined in Swiss Patent 1339/90-7. These are especially the compounds of formula (I) 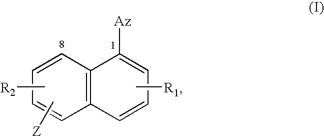
wherein
the dotted line denotes an additional bond or no additional bond;
Az is imidazolyl, triazolyl or tetrazolyl bonded via a ring nitrogen atom, each of those radicals being unsubstituted or substituted at carbon atoms by lower alkyl or by aryl-lower alkyl;
Z is carboxy, lower alkoxycarbonyl, carbamoyl, N-lower alkylcarbamoyl, N,N-di-lower alkylcarbamoyl, N-arylcarbamoyl, cyano, halogen, hydroxy, lower alkoxy, aryl-lower alkoxy, aryloxy, lower alkyl, trifluoromethyl or aryl-lower alkyl; and
R₁ and R₂, independently of one another, are each hydrogen, lower alkyl, lower alkoxy, hydroxy, halogen or trifluoromethyl; aryl being phenyl or naphthyl each of which is unsubstituted or substituted by one or two substituents from the group consisting of lower alkyl, lower alkoxy, hydroxy, halogen and trifluoromethyl, with the proviso that neither Z nor R₂ is hydroxy in the 8-position;
   and pharmaceutically acceptable salts thereof.

Preferred compounds of this group are:
1. 6-cyano-1-(1-imidazolyl)-3,4-dihydronaphthalene;
2. 6-cyano-1-[1-(1,2,4-triazolyl)]-3,4-dihydronaphthalene;
3. 6-chloro-1-(1-imidazolyl)-3,4-dihydronaphthalene; and
4. 6-bromo-1-(1-imidazolyl)-3,4-dihydronaphthalene.

(f) The compounds of formula (I), as defined in Swiss Patent 3014/90-0. These are especially the compounds of formula (I) 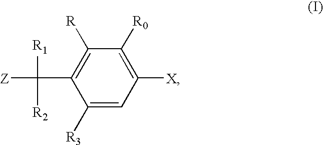
wherein
Z is a five-membered nitrogen-containing heteroaromatic ring selected from the group 5-isothiazolyl, 5-thiazolyl, 5-isoxazolyl, 5-oxazolyl, 5-(1,2,3-thiadiazolyl), 5-(1,2,3-oxadiazolyl), 3-(1,2,5-thiadiazolyl), 3-(1,2,5-oxadiazolyl), 4-isothiazolyl, 4-isoxazolyl, 4-(1,2,3-thiadiazolyl), 4-(1,2,3-oxadiazolyl), 2-(1,3,4-thiadiazolyl), 2-(1,3,4-oxadiazolyl), 5-(1,2,4-thiadiazolyl) and 5-(1,2,4-oxadiazolyl);
R and R₀ are hydrogen, or
R and R₀, together, are a benzo group that is unsubstituted or substituted by lower alkyl, lower alkoxy, hydroxy, halogen or by trifluoromethyl;
R₁ is hydrogen, hydroxy, chlorine or fluorine;
R₃ is hydrogen;
R₂ is hydrogen, lower alkyl or phenyl that is unsubstituted or substituted by lower alkyl, lower alkoxy, hydroxy, halogen, trifluoromethyl or by cyano, or
R₁ and R₂, together, are methylidene, or
R₂ and R₃, together, are —(CH₂)₃—, or
R₁ and R₂ and R₃, together, are a group ═CH—(CH₂)₂—, wherein the single bond is linked to the benzene ring;
X is cyano and X may also be halogen when R₂ and R₃, together, are —(CH₂)₃—, or
R₁ and R₂ and R₃, together, are a group ═CH—(CH₂)₂—;
   and pharmaceutically acceptable salts thereof.

Preferred compounds of this group are:
1. 4-[α-(4-cyanophenyl)-α-hydroxy-5-isothiazolylmethyl]-benzonitrile;
2. 4-[α-(4-cyanophenyl)-5-isothiazolylmethyl]-benzonitrile;
3. 4-[α-(4-cyanophenyl)-5-thiazolylmethyl]-benzonitrile;
4. 1-(4-cyanophenyl)-1-(5-thiazolyl)-ethylene;
5. 6-cyano-1-(5-isothiazolyl)-3,4-dihydronaphthalene; and
6. 6-cyano-1-(5-thiazolyl)-3,4-dihydronaphthalene.

(g) The compounds of formula (VI), as defined in Swiss Patent 3014/90-0. These are especially the compounds of formula (VI) 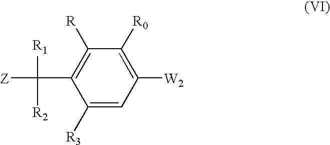
wherein
Z is a five-membered nitrogen-containing heteroaromatic ring selected from the group 5-isothiazolyl, 5-thiazolyl, 5-isoxazolyl, 5-oxazolyl, 5-(1,2,3-thiadiazolyl), 5-(1,2,3-oxadiazolyl), 3-(1,2,5-thiadiazolyl), 3-(1,2,5-oxadiazolyl), 4-isothiazolyl, 4-isoxazolyl, 4-(1,2,3-thiadiazolyl), 4-(1,2,3-oxadiazolyl), 2-(1,3,4-thiadiazolyl), 2-(1,3,4-oxadiazolyl), 5-(1,2,4-thiadiazolyl) and 5-(1,2,4-oxadiazolyl);
R and R₀ are each hydrogen, or
R and R₀, together, are a benzo group that is unsubstituted or substituted by lower alkyl, lower alkoxy, hydroxy, halogen or by trifluoromethyl;
R₁ is hydrogen, hydroxy, chlorine or fluorine;
R₃ is hydrogen;
R₂ is hydrogen, lower alkyl or phenyl that is unsubstituted or substituted by lower alkyl, lower alkoxy, hydroxy, halogen, trifluoromethyl, aryl-lower alkoxy or by aryloxy, or
R₁ and R₂, together, are methylidene; and
W₂ is halogen, hydroxy, lower alkoxy, aryl-lower alkoxy or aryloxy, aryl in each case being phenyl that is unsubstituted or substituted by lower alkyl, lower alkoxy, hydroxy, halogen or by trifluoromethyl;
   and pharmaceutically acceptable salts thereof.

Preferred compounds of this group are:
1. bis(4,4′-bromophenyl)-(5-isothiazolyl)methanol;
2. bis(4,4′-bromophenyl)-(5-isothiazolyl)methane;
3. bis(4,4′-bromophenyl)-(5-thiazolyl)methanol; and
4. bis(4,4′-bromophenyl)-(5-thiazolyl)methane.

(h) The compounds of formula (I), as defined in Swiss Patent 3923/90-4. These are especially the compounds of formula (I) 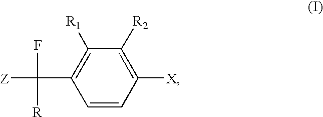
wherein
Z is imidazolyl, triazolyl, tetrazolyl, pyrrolyl, pyrazolyl, indolyl, isoindolyl, benzimidazolyl, benzopyrazolyl, benzotriazolyl, pyridyl, pyrimidyl, pyrazinyl, pyridazinyl, triazinyl, quinolinyl or isoquinolinyl, all those radicals being bonded via their heterocyclic rings and all those radicals being unsubstituted or substituted by lower alkyl, hydroxy, lower alkoxy, halogen or by trifluoromethyl;
R₁ and R₂, independently of one another, are each hydrogen or lower alkyl, or
R₁ and R₂, together, are C₃-C₄alkylene or a benzo group that is unsubstituted or substituted as indicated below for aryl;
R is hydrogen, lower alkyl, aryl or heteroaryl;
X is cyano, carbamoyl, N-lower alkylcarbamoyl, N,N-di-lower alkylcarbamoyl, N,N-lower alkylenecarbamoyl, N,N-lower alkylenecarbamoyl interrupted by —O—, —S— or —NR″—, wherein R″ is hydrogen, lower alkyl or lower alkanoyl, N-cycloalkylcarbamoyl, N-(lower alkyl-substituted cycloalkyl)-carbamoyl, N-cycloalkyl-lower alkylcarbamoyl, N-(lower alkyl-substituted cycloalkyl)-lower alkylcarbamoyl, N-aryl-lower alkylcarbamoyl, N-arylcarbamoyl, N-hydroxycarbamoyl, hydroxy, lower alkoxy, aryl-lower alkoxy or aryloxy and is also halogen when Z is imidazolyl, triazolyl, tetrazolyl, pyrrolyl, pyrazolyl, indolyl, isoindolyl, benzimidazolyl, benzopyrazolyl or benzotriazolyl, wherein aryl is phenyl or naphthyl; these radicals being unsubstituted or substituted by from 1-4 substituents from the group consisting of lower alkyl, lower alkenyl, lower alkynyl, lower alkylene (linked to two adjacent carbon atoms), C₃-C₈cycloalkyl, phenyl-lower alkyl, phenyl; lower alkyl that is substituted in turn by hydroxy, lower alkoxy, phenyl-lower alkoxy, lower alkanoyloxy, halogen, amino, lower alkylamino, di-lower alkylamino, mercapto, lower alkylthio, lower alkylsulfinyl, lower alkylsulfonyl, carboxy, lower alkoxycarbonyl, carbamoyl, N-lower alkylcarbamoyl, N,N-di-lower alkylcarbamoyl and/or by cyano, hydroxy, lower alkoxy, halo-lower alkoxy, phenyl-lower alkoxy, phenoxy, lower alkenyloxy, halo-lower alkenyloxy, lower alkynyloxy, lower alkylenedioxy (linked to two adjacent carbon atoms), lower alkanoyloxy, phenyl-lower alkanoyloxy, phenylcarbonyloxy, mercapto, lower alkylthio, phenyl-lower alkylthio, phenylthio, lower alkylsulfinyl, phenyl-lower alkylsulfinyl, phenylsulfinyl, lower alkylsulfonyl, phenyl-lower alkylsulfonyl, phenylsulfonyl, halogen, nitro, amino, lower alkylamino, C₃-C₈cycloalkylamino, phenyl-lower alkylamino, phenylamino, di-lower alkylamino, N-lower alkyl-N-phenylamino, N-lower alkyl-N-phenyl-lower alkylamino, lower alkyleneamino or lower alkyleneamino interrupted by —O—, —S— or —NR″—, wherein R″ is hydrogen, lower alkyl or lower alkanoyl, lower alkanoylamino, phenyl-lower alkanoylamino, phenylcarbonylamino, lower alkanoyl, phenyl-lower alkanoyl, phenylcarbonyl, carboxy, lower alkoxycarbonyl, carbamoyl, N-lower alkylcarbamoyl, N,N-di-lower alkylcarbamoyl, N,N-lower alkylenecarbamoyl, N,N-lower alkylenecarbamoyl interrupted by —O—, —S— or —NR″—, wherein R″ is hydrogen, lower alkyl or lower alkanoyl, N-cycloalkylcarbamoyl, N-(lower alkyl-substituted cycloalkyl)-carbamoyl, N-cycloalkyl-lower alkylcarbamoyl, N-(lower alkyl-substituted cycloalkyl)-lower alkylcarbamoyl, N-hydroxycarbamoyl, N-phenyl-lower alkylcarbamoyl, N-phenylcarbamoyl, cyano, sulfo, lower alkoxysulfonyl, sulfamoyl, N-lower alkylsulfamoyl, N,N-di-lower alkylsulfamoyl and N-phenylsulfamoyl; the phenyl groups occurring in the substituents of phenyl and naphthyl in turn being unsubstituted or substituted by lower alkyl, lower alkoxy, hydroxy, halogen and/or by trifluoromethyl, wherein heteroaryl is indolyl, isoindolyl, benzimidazolyl, benzopyrazolyl, benzotriazolyl, benzo[b]furanyl, benzo[b]thienyl, benzoxazolyl or benzothiazolyl, those radicals being unsubstituted or substituted by from 1-3 identical or different substituents selected from lower alkyl, hydroxy, lower alkoxy, halogen, cyano and trifluoromethyl;
   and pharmaceutically acceptable salts thereof.

Those compounds are especially the compounds of formula (I),

wherein

Z is 1-imidazolyl, 1-(1,2,4-triazolyl), 1-(1,3,4-triazolyl), 1-(1,2,3-triazolyl), 1-tetrazolyl, 2-tetrazolyl, 3-pyridyl, 4-pyridyl, 4-pyrimidyl, 5-pyrimidinyl or 2-pyrazinyl;
R₁ and R₂, independently of one another, are each hydrogen or lower alkyl, or
R₁ and R₂, together, are 1,4-butylene or a benzo group;
R is lower alkyl; phenyl that is unsubstituted or substituted by cyano, carbamoyl, halogen, lower alkyl, trifluoromethyl, hydroxy, lower alkoxy or by phenoxy; or benzotriazolyl or benzo[b]furanyl; the last two radicals being unsubstituted or substituted by from 1-3 identical or different substituents selected from lower alkyl, halogen and cyano; and
X is cyano or carbamoyl and is also halogen when Z is 1-imidazolyl, 1-(1,2,4-triazolyl), 1-(1,3,4-triazolyl), 1-(1,2,3-triazolyl), 1-tetrazolyl or 2-tetrazolyl;
   and pharmaceutically acceptable salts thereof.

Preferred compounds of this group are:
1. 4-[α-(4-cyanophenyl)-α-fluoro-1-(1,2,4-triazolyl)methyl]-benzonitrile;
2. 4-[α-(4-cyanophenyl)-α-fluoro-(2-tetrazolyl)methyl]-benzonitrile;
3. 4-[α-(4-cyanophenyl)-α-fluoro-(1-tetrazolyl)methyl]-benzonitrile;
4. 4-[α-(4-cyanophenyl)-α-fluoro-(1-imidazolyl)methyl]-benzonitrile;
5. 1-methyl-6-[α-(4-chlorophenyl)-α-fluoro-1-(1,2,4-triazolyl)methyl]-benzotriazole;
6. 4-[α-(4-cyanophenyl)-α-fluoro-1-(1,2,3-triazolyl)methyl]-benzonitrile;
7. 7-cyano-4-[α-(4-cyanophenyl)-α-fluoro-1-(1,2,4-triazolyl)methyl]-2,3-dimethylbenzo[b]furan;
8. 4-[α-(4-bromophenyl)-α-fluoro-1-(1,2,4-triazolyl)methyl]-benzonitrile;
9. 4-[α-(4-cyanophenyl)-α-fluoro-(5-pyrimidyl)methyl]-benzonitrile;
10. 4-[α-(4-bromophenyl)-α-fluoro-(5-pyrimidyl)methyl]-benzonitrile;
11. 4-[α-(4-cyanophenyl]-α-fluoro-(3-pyridyl)methyl]-benzonitrile;
12. 7-bromo-4-[α-(4-cyanophenyl)-α-fluoro-(1-imidazolyl)methyl]-2,3-dimethylbenzo[b]furan;
13. 7-bromo-4-[α-(4-cyanophenyl)-α-fluoro-1-(1,2,4-triazolyl)methyl]-2,3-dimethylbenzo[b]furan;
14. 4-[α-(4-cyanophenyl)-α-fluoro-(5-pyrimidyl)methyl]-benzonitrile;
15. 4-[α-(4-bromophenyl)-α-fluoro-(5-pyrimidyl)methyl]-benzonitrile;
16. 4-[α-(4-cyanophenyl)-1-(1,2,3-triazolyl)methyl]-benzonitrile;
17. 2,3-dimethyl-4-[α-(4-cyanophenyl)-1-(1,2,4-triazolyl)methyl]-7-cyano-benzo[b]furan;
18. 4-[α-(4-cyanophenyl)-(5-pyrimidyl)methyl]-benzonitrile;
19. 4-[α-(4-bromophenyl)-(5-pyrimidyl)methyl]-benzonitrile;
20. 2,3-dimethyl-4-[α-(4-cyanophenyl)-(1-imidazolyl)methyl]-7-bromo-benzo[b]furan; and
21. 2,3-dimethyl-4-[α-(4-cyanophenyl)-1-(1,2,4-triazolyl)methyl]-7-bromo-benzo[b]furan.

(i) The compounds of formula (I), as defined in EP A 114 033. These are especially the compounds of formula (I) 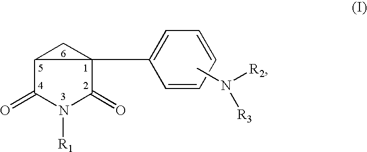
wherein
R₁ is hydrogen;
R₂ is hydrogen, sulfo, C₁-C₇alkanoyl or C₁-C₇alkanesulfonyl; and
R₃ is hydrogen, or
R₁ is C₁-C₁₂alkyl, C₂-C₁₂alkenyl, C₂-C₇alkynyl, C₃-C₁₀cycloalkyl, C₃-C₁₀cycloalkenyl, C₃-C₆cycloalkyl-C₁-C₄alkyl, C₃-C₆cycloalkylC₂-C₄alkenyl or C₃-C₆cycloalkenylC₁-C₄alkyl;
R₂ is hydrogen, C₁-C₇alkyl, sulfo, C₁-C₇alkanoyl or C₁-C₇alkanesulfonyl; and
R₃ is hydrogen or C₁-C₇alkyl;
   and pharmaceutically acceptable salts of those compounds.

Preferred compounds of this group are:
1. 1-(4-aminophenyl)-3-methyl-3-azabicyclo[3.1.0]hexane-2,4-dione;
2. 1-(4-aminophenyl)-3-n-propyl-3-azabicyclo[3.1.0]hexane-2,4-dione;
3. 1-(4-aminophenyl)-3-isobutyl-3-azabicyclo[3.1.0]hexane-2,4-dione;
4. 1-(4-aminophenyl)-3-n-heptyl-3-azabicyclo[3.1.0]hexane-2,4-dione; and
5. 1-(4-aminophenyl)-3-cyclohexylmethyl-3-azabicyclo[3.1.0]hexane-2,4-dione.

(j) The compounds of formula (I), as defined in EP A 166 692. These are especially the compounds of formula (I) 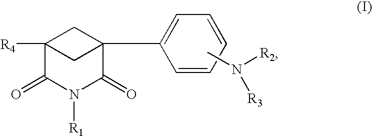
wherein
R₁ is hydrogen, alkyl having from 1-12 carbon atoms, alkenyl having from 2-12 carbon atoms, lower alkynyl, cycloalkyl or cycloalkenyl each having from 3-10 carbon atoms, cycloalkyl-lower alkyl having from 4-10 carbon atoms, cycloalkyl-lower alkenyl having from 5-10 carbon atoms, cycloalkenyl-lower alkyl having from 4-10 carbon atoms or aryl having from 6-12 carbon atoms or aryl-lower alkyl having from 7-15 carbon atoms, each of which is unsubstituted or substituted by lower alkyl, hydroxy, lower alkoxy, acyloxy, amino, lower alkylamino, di-lower alkylamino, acylamino or by halogen;
R₂ is hydrogen, lower alkyl, sulfo, lower alkanoyl or lower alkanesulfonyl;
R₃ is hydrogen or lower alkyl; and
R₄ is hydrogen, lower alkyl, phenyl or phenyl substituted by —N(R₂)(R₃);
   and pharmaceutically acceptable salts thereof, radicals described as “lower” containing up to and including 7 carbon atoms.

Preferred compounds of this group are:
1. 1-(4-aminophenyl)-3-n-propyl-3-azabicyclo[3.1.1]heptane-2,4-dione;
2. 1-(4-aminophenyl)-3-methyl-3-azabicyclo[3.1.1]heptane-2,4-dione;
3. 1-(4-aminophenyl)-3-n-decyl-3-azabicyclo[3.1.1]heptane-2,4-dione;
4. 1-(4-aminophenyl)-3-cyclohexyl-3-azabicyclo[3.1.1]heptane-2,4-dione; and
5. 1-(4-aminophenyl)-3-cyclohexylmethyl-3-azabicyclo[3.1.1]heptane-2,4-dione.

(k) The compounds of formula (I), as defined in EP A 356 673. These are especially the compounds of formula (I) 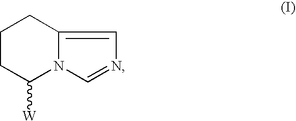
wherein
W is (α) is a 2-naphthyl or 1-anthryl radical, wherein each benzene ring is unsubstituted or substituted by a substituent selected from halogen, hydroxy, carboxy, cyano and nitro, or
   (α) is 4-pyridyl, 2-pyrimidyl or 2-pyrazinyl, each of those radicals being unsubstituted or substituted by a substituent selected from halogen, cyano, nitro, C₁-C₄alkoxy and C₂-C₅alkoxycarbonyl;
      and pharmaceutically acceptable salts thereof.

Preferred compounds of this group are:
1. 5-(2′-naphthyl)-5,6,7,8-tetrahydroimidazo[1,5-a]pyridine; and
2. 5-(4′-pyridyl)-5,6,7,8-tetrahydroimidazo[1,5-a]pyridine.

(l) The compounds of formula (I) or (Ia), as defined in EP A 337 929. These are especially the compounds of formula (I/Ia) 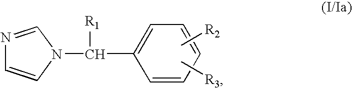
wherein
R₁ is hydrogen, methyl, ethyl, propyl, propenyl, isopropyl, butyl, hexyl, octyl, decyl, cyclopentyl, cyclohexyl, cyclopentylmethyl, cyclohexylmethyl or benzyl;
R₂ is benzyloxy, 3-bromo-, 4-bromo-, 4-chloro-, 2,3-, 2,4-, 4,5- or 4,6-dichloro-benzyloxy; and
R₃ is cyano; C₂-C₁₀alkanoyl that is unsubstituted or mono- or poly-substituted by halogen, methoxy, amino, hydroxy and/or by cyano; benzoyl that is unsubstituted or substituted by one or more substituents from the group halogen, C₁-C₄alkyl, methoxy, amino, hydroxy and cyano; carboxy, (methoxy, ethoxy or butoxy)-carbonyl, carbamoyl, N-isopropylcarbamoyl, N-phenylcarbamoyl, N-pyrrolidylcarbonyl, nitro or amino;
   and pharmaceutically acceptable salts thereof.

Preferred compounds of this group are:
1. 4-(2,4-dichlorobenzyloxy)-3-[1-(1-imidazolyl)-butyl]-benzonitrile;
2. (4-(4-bromobenzyloxy)-3-[1-(1-imidazolyl)-butyl]-phenyl pentyl ketone;
3. 4-(4-bromobenzyloxy)-3-[1-(1-imidazolyl)-butyl]-benzanilide;
4. 4-(4-bromobenzyloxy)-3-[1-(1-imidazolyl)-butyl]-benzoic acid;
5. 3-(2,4-dichlorobenzyloxy)-4-[1-(1-imidazolyl)-butyl]-benzonitrile;
6. 3-(2,4-dichlorobenzyloxy)-4-[1-(1-imidazolyl)-butyl]-benzoic acid methyl ester;
7. 3-(2,4-dichlorobenzyloxy)-4-[1-(1-imidazolyl)-butyl]-benzoic acid;
8. 3-(3-bromobenzyloxy)-4-[1-(1-imidazolyl)-butyl]-benzonitrile;
9. 4-(3-bromobenzyloxy)-3-[1-(1-imidazolyl)-butyl]-benzonitrile;
10. 3-(4-bromobenzyloxy)-4-[1-(1-imidazolyl)-butyl]-benzoic acid;
11. 3-(4-bromobenzyloxy)-4-[1-(1-imidazolyl)-butyl]-benzanilide;
12. 3-(4-bromobenzyloxy)-4-[1-(1-imidazolyl)-butyl]-phenyl pentyl ketone;
13. 4-(4-bromobenzyloxy)-3-[1-(1-imidazolyl)-butyl]-benzonitrile;
14. 3-(4-bromobenzyloxy)-4-(1-(1-imidazolyl)-butyl]-benzonitrile;
15. 4-nitro-2-[1-(1-imidazolyl)-butyl]-phenyl-(2,4-dichlorobenzyl) ether;
16. 4-amino-2-[1-(1-imidazolyl)-butyl]-phenyl-(2,4-dichlorobenzyl) ether; and
17. (2,4-dichlorobenzyl)-[2-(1-imidazolyl-methyl)₄-nitrophenyl] ether.

(m) The compounds of formula (I), as defined in EP A 337 928. These are especially the compounds of formula (I) 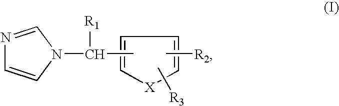
wherein
R₁ is hydrogen, methyl, ethyl, propyl, propenyl, isopropyl, butyl, hexyl, octyl, decyl, cyclopentyl, cyclohexyl, cyclopentylmethyl, cyclohexylmethyl or benzyl;
R₂ is hydrogen, halogen, cyano, methyl, hydroxymethyl, cyanomethyl, methoxymethyl, pyrrolidinylmethyl, carboxy, (methoxy, ethoxy or butoxy)-carbonyl, carbamoyl, N-isopropylcarbamoyl, N-phenylcarbamoyl, N-pyrrolidylcarbonyl; C₂-C₁₀alkanoyl that is unsubstituted or mono- or poly-substituted by halogen, methoxy, ethoxy, amino, hydroxy and/or by cyano; or benzoyl that is unsubstituted or substituted by one or more substituents from the group halogen, C₁-C₄alkyl, methoxy, ethoxy, amino, hydroxy and cyano, R₃ is hydrogen, benzyloxy, 3-bromo-, 4-bromo-, 4-chloro-, 2,3-, 2,4-, 4,5- or 4,6-dichloro-benzyloxy; and
X is —CH═N—, —CH═N(—O)— or —S—;
   and pharmaceutically acceptable salts thereof.

Preferred compounds of this group are:
1. 5-(1-(1-imidazolyl)-butyl]-thiophene-2-carbonitrile;
2. 2-[1-(1-imidazolyl)-butyl]-thiophene-4-carbonitrile;
3. 2-[1-(1-imidazolyl)-butyl]4-bromo-thiophene;
4. 2-[1-(1-imidazolyl)-butyl]-5-bromo-thiophene;
5. 5-[1-(1-imidazolyl)-butyl]-2-thienyl pentyl ketone;
6. 5-[1-(1-imidazolyl)-butyl]-2-thienyl ethyl ketone;
7. 5-(4-chlorobenzyloxy)-4-[1-(1-imidazolyl)-pentyl]-pyridine-2-carbonitrile;
8. 3-(4-chlorobenzyloxy)-4-[1-(1-imidazolyl)-pentyl]-pyridine-2-carbonitrile;
9. 3-(4-chlorobenzyloxy)-4-[1-(1-imidazolyl)-pentyl]-pyridine-N-oxide; and
10. 3-(4-chlorobenzyloxy)-4-[1-(1-imidazolyl)-pentyl]-pyridine.

(n) The compounds of formula (I), as defined in EP A 340 153. These are especially the compounds of formula (I) 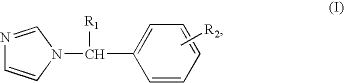
wherein
R₁ is hydrogen, methyl, ethyl, propyl, propenyl, isopropyl, butyl, hexyl, octyl, decyl, cyclopentyl, cyclohexyl, cyclopentylmethyl, cyclohexylmethyl or benzyl; and
R₂ is a radical from the group methyl, ethyl, propyl, benzyl, phenyl and ethenyl that is substituted by hydroxy, cyano, methoxy, butoxy, phenoxy, amino, pyrrolidinyl, carboxy, lower alkoxycarbonyl or by carbamoyl, or
R₂ is formyl or derivatised formyl that can be obtained by reaction of the formyl group with an amine or amine derivative from the group hydroxylamine, O-methylhydroxylamine, O-ethylhydroxylamine, O-allylhydroxylamine, O-benzylhydroxylamine, O-4-nitrobenzyloxyhydroxylamine, 0-2,3,4,5,6-pentafluorobenzyloxyhydroxylamine, semicarbazide, thiosemicarbazide, ethylamine and aniline; acetyl; propionyl; butyryl; valeryl; caproyl; benzoyl that is unsubstituted or substituted by one or more substituents from the group halogen, C₁-C₄alkyl, methoxy, amino, hydroxy and cyano; carboxy, (methoxy, ethoxy or butoxy)-carbonyl, carbamoyl, N-isopropylcarbamoyl, N-phenylcarbamoyl or N-pyrrolidylcarbonyl;
   and pharmaceutically acceptable salts thereof.

Preferred compounds of this group are:
1. 4-(1-(1-imidazolyl)-butyl)-benzoic acid methyl ester;
2. 4-(1-(1-imidazolyl)-butyl)-benzoic acid butyl ester;
3. 4-(1-(1-imidazolyl)-butyl)-phenyl-acetonitrile;
4. 4-(1-(1-imidazolyl)-butyl)-benzaldehyde;
5. 4-(1-(1-imidazolyl)-butyl)-benzyl alcohol;
6. {4-[1-(1-imidazolyl)-butyl]-phenyl}-2-propyl ketone;
7. 4-[1-(1-imidazolyl)-butyl]-phenyl propyl ketone;
8. 4-[1-(1-imidazolyl)-butyl]-phenyl butyl ketone;
9. 4-[1-(1-imidazolyl)-butyl]-phenyl pentyl ketone; and
10. 4-[1-(1-imidazolyl)-butyl]-phenyl hexyl ketone.

(o) The compounds of formula (I), as defined in DE A 4 014 006. These are especially the compounds of formula (I) 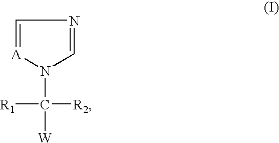
wherein
A is an N-atom or a CH radical; and
W is a radical of the formula 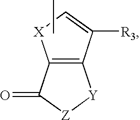
   wherein
X is an oxygen or a sulfur atom or a —CH═CH— group;
Y is a methylene group, an oxygen or a sulfur atom; and
Z is a —(CH₂)ₙ— group, wherein n=1, 2 or 3 and either
   a) R₃ in W is a hydrogen atom; and
      R₁ and R₂, independently of one another, are each a hydrogen atom, a C₁-C₁₀alkyl group or a C₃-C₇cycloalkyl group, or
   b) R₂ is as defined under a); and
      R₁, together with R₃, forms a —(CH₂)ₘ— group, wherein m=2, 3 or 4;
         and their pharmaceutically acceptable addition salts with acids.

Preferred compounds of this group are:
1. 5-[1-(1-imidazolyl)-butyl]-1-indanone,
2. 7-[1-(1-imidazolyl)-butyl]-1-indanone,
3. 6-[1-(1-imidazolyl)-butyl]-1-indanone,
4. 6-(1-imidazolyl)-6,7,8,9-tetrahydro-1H-benz[e]inden-3(2H)-one;
5. 2-[1-(1-imidazolyl)-butyl]-4,5-dihydro-6-oxo-cyclopenta[b]-thiophene;
6. 6-[1-(1-imidazolyl)-butyl]-3,4-dihydro-2H-naphthalen-1-one;
7. 2-[1-(1-imidazolyl)-butyl]-6,7-dihydro-5H-benzo[b]thiophen-4-one;
8. 6-[1-(1-imidazolyl)-butyl]-2H-benzo[b]furan-3-one;
9. 5-[cyclohexyl-(1-imidazolyl)-methyl]-1-indanone;
10. 2-[1-(1-imidazolyl)-butyl]-4,5-dihydro-6H-benzo[b]thiophen-7-one;
11. 5-[1-(1-imidazolyl)-1-propyl-butyl]-1-indanone;
12. 2-[1-(1-imidazolyl)-butyl]4,5-dihydro-6H-benzo[b]thiophen-7-one;
13. 2-[1-(1-imidazolyl)-butyl]4,5-dihydro-6-oxo-cyclopenta[b]-thiophene;
14. 5-(1-imidazolylmethyl)-1-indanone; and
15. 5-[1-(1,2,4-triazolyl)-methyl]-1-indanone.

(p) The compounds of formula (I), as disclosed in DE A 3 926 365. These are especially the compounds of formula (I) 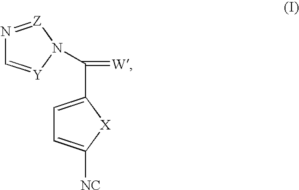
wherein
W′ is a cyclopentylidene, cyclohexylidene, cycloheptylidene or 2-adamantylidene radical;
X is the grouping —CH═CH—, an oxygen or a sulfur atom; and
Y and Z, independently of one another, are each a methine group (CH) or a nitrogen atom; and their pharmaceutically acceptable addition salts with acids.

Preferred compounds of this group are:
1. 4-[1-cyclohexylidene-1-(imidazolyl)-methyl]-benzonitrile;
2. 4-[1-cyclopentylidene-1-(imidazolyl)-methyl]-benzonitrile;
3. 4-[1-cycloheptylidene-1-(imidazolyl)-methyl]-benzonitrile;
4. 4-[2-adamantylidene-1-(imidazolyl)-methyl]-benzonitrile;
5. 4-[1-cyclohexylidene-1-(1,2,4-triazolyl)-methyl]-benzonitrile;
6. 4-[1-cyclopentylidene-1-(1,2,4-triazolyl)-methyl]-benzonitrile;
7. 4-[1-cycloheptylidene-1-(1,2,4-triazolyl)-methyl]-benzonitrile;
8. 4-[2-adamantylidene-1-(1,2,4-triazolyl)-methyl]-benzonitrile;
9. 4-[1-cyclohexylidene-1-(1,2,3-triazolyl)-methyl]-benzonitrile;
10. 4-[1-cyclopentylidene-1-(1,2,3-triazolyl)-methyl]-benzonitrile; and
11. 5-[cyclohexylidene-1-imidazolylmethyl]-thiophene-2-carbonitrile.

(q) The compounds of formula (I), as defined in DE A 3 740 125. These are especially the compounds of formula (I) 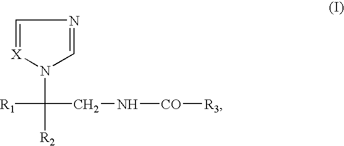
wherein
X is CH or N;
R₁ and R₂ are identical or different and are each phenyl or halophenyl; and
R₃ is C₁-C₄alkyl; C₁-C₄alkyl substituted by CN, C₁-C₄alkoxy, benzyloxy or by C₁-C₄alkoxy-(mono-, di- or tri-)ethyleneoxy; C₁-C₄alkoxy; phenyl; phenyl that is substituted by halogen or by cyano; a C₅-C₇cycloalkyl group that is optionally condensed by benzene, or is thienyl, pyridyl or 2- or 3-indolyl;
   and pharmaceutically acceptable acid addition salts thereof.

A preferred compound of this group is 2,2-bis(4-chlorophenyl)-2-(1H-imidazol-1-yl)-1-(4-chlorobenzoyl-amino)ethane.

(r) The compounds of formula (I), as defined in EP A 293978. These are especially the compounds of formula (I) 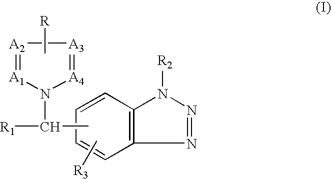
pharmaceutically acceptable salts and stereochemically isomeric forms thereof,
wherein
-A₁=A₂-A₃=A₄- is a divalent radical selected from —CH═N—CH═CH—, —CH═N—CH═N— and —CH═N—N═CH—;
R is hydrogen or C₁-C₆alkyl;
R₁ is hydrogen, C₁-C₁₀alkyl, C₃-C₇cycloalkyl, Ar₁, Ar₂-C₁-C₆alkyl, C₂-C₆alkenyl or C₂-C₆alkynyl;
R₂ is hydrogen, C₁-C₁₀alkyl that is unsubstituted or substituted by Ar₁, C₃-C₇cycloalkyl, hydroxy, C₁-C₆alkoxy, Ar₁, C₂-C₆alkenyl, C₂-C₆alkynyl, C₃-C₇cycloalkyl, bicyclo[2.2.1]heptan-2-yl, 2,3-dihydro-1H-indenyl, 1,2,3,4-tetrahydronaphthyl, hydroxy, C₂-C₆alkenyloxy that is unsubstituted or substituted by Ar₂, C₂-C₆alkynyloxy, pyrimidyloxy, di(Ar₂)methoxy, (1-C₁-C₄alkyl-4-piperidinyl)oxy, C₁-C₁₀alkoxy; or C₁-C₁₀alkoxy that is substituted by halogen, hydroxy, C₁-C₆alkyloxy, amino, mono- or di-(C₁-C₆alkyl)amino, trifluoromethyl, carboxy, C₁-C₆alkoxycarbonyl, Ar₁, Ar₂-O—, Ar₂-S—, C₃-C₇cycloalkyl, 2,3-dihydro-1,4-benzodioxinyl, 1H-benzimidazolyl, C₁-C₄alkyl-substituted 1H-benzimidazolyl, (1,1′-biphenyl)-4-yl or by 2,3-dihydro-2-oxo-1H-benzimidazolyl; and
R₃ is hydrogen, nitro, amino, mono- or di-(C₁-C₆alkyl)amino, halogen, C₁-C₆alkyl, hydroxy or C₁-C₆alkoxy,
wherein
   Ar₁ is phenyl, substituted phenyl, naphthyl, pyridyl, aminopyridyl, imidazolyl, triazolyl, thienyl, halothienyl, furanyl, C₁-C₆alkylfuranyl, halofuranyl or thiazolyl;
   Ar₂ is phenyl, substituted phenyl or pyridyl, and
   “substituted phenyl” is phenyl that is substituted by up to 3 substituents in each case selected independently of one another from the group consisting of halogen, hydroxy, hydroxymethyl, trifluoromethyl, C₁-C₆alkyl, C₁-C₆alkoxy, C₁-C₆alkoxycarbonyl, carboxy, formyl, hydroxyiminomethyl, cyano, amino, mono- and di-(C₁-C₆alkyl)amino and nitro.

Preferred compounds of this group are:
1. 6-[(1H-imidazol-1-yl)-phenylmethyl]-1-methyl-1H-benzotriazole; and
2. 6-[(4-chlorophenyl)(1H-1,2,4-triazol-1-yl)methyl]-1-methyl-1H-benzotriazole.

(s) The compounds of formula (II), as defined in EP A 250198, especially
1. 2-(4-chlorophenyl)-1,1-di(1,2,4-triazol-1-ylmethyl)ethanol;
2. 2-(4-fluorophenyl)-1,1-di(1,2,4-triazol-1-ylmethyl)ethanol;
3. 2-(2-fluoro-4-trifluoromethylphenyl)-1,1-di(1,2,4-triazol-1-ylmethyl)ethanol;
4. 2-(2,4-dichlorophenyl)-1,1-di(1,2,4-triazol-1-ylmethyl)ethanol;
5. 2-(4-chlorophenyl)-1,1-di(1,2,4-triazol-1-ylmethyl)-ethanol; and
6. 2-(4-fluorophenyl)-1,1-di(1,2,4-triazol-1-yl-methyl)ethanol.

(t) The compounds of formula (I), as defined in EP A 281283, especially
1. (1R*,2R*)-6-fluoro-2-(4-fluorophenyl)-1,2,3,4-tetrahydro-1-(1H-1,2,4-triazol-1-ylmethyl)naphthalene;
2. (1R*,2R*)-6-fluoro-2-(4-fluorophenyl)-1,2,3,4-tetrahydro-1-(1H-imidazolylmethyl)naphthalene;
3. (1R*,2R*)- and (1R,2S)-2-(4-fluorophenyl)-1,2,3,4-tetrahydro-1-(1H-1,2,4-triazol-1-ylmethyl)naphthalene-6-carbonitrile;
4. (1R*,2R*)- and (1R*,2S)-2-(4-fluorophenyl)-1,2,3,4-tetrahydro-1-(1H-imidazolylmethyl)naphthalene-6-carbonitrile;
5. (1R*,2R*)- and (1R*,2S)-1,2,3,4-tetrahydro-1-(1H-1,2,4-triazol-1-ylmethyl)naphthalene-2,6-dicarbonitrile;
6. (1R*,2R*)- and (1R*,2S*)-1,2,3,4-tetrahydro-1-(1H-imidazol-1-ylmethyl)naphthalene-2,6-dicarbonitrile; and

7. (1R*,2S)-2-(4-fluorophenyl)-1,2,3,4-tetrahydro-1-(5-methyl-1H-imidazolylmethyl)naphthalene-6-carbonitrile.

(u) The compounds of formula (I), as defined in EP A 296749, especially
1. 2,2′-[5-(1H-1,2,4-triazol-1-ylmethyl)-1,3-phenylene]di(2-methylpropiononitrile);
2. 2,2′-[5-(imidazol-1-ylmethyl)-1,3-phenylene]di(2-methylpropiononitrile);
3. 2-[3-(1-hydroxy-1-methylethyl)-5-(5H-1,2,4-triazol-1-ylmethyl)phenyl]-2-methylpropiononitrile;
4. 2,2′-[5-dideuterio(1H-1,2,4-triazol-1-yl)methyl-1,3-phenylene]di(2-trideuteriomethyl-3,3,3-trideuteriopropiononitrile); and
5. 2,2′-[5-dideuterio(1H-1,2,4-triazol-1-yl)methyl-1,3-phenylene]di(2-methylpropiononitrile).

(v) The compounds of formula (I), as defined in EP A 299683, especially
1. (Z)-α-(1,2,4-triazol-1-ylmethyl)stilbene-4,4′-dicarbonitrile;
2. (Z)-4′-chloro-α-(1,2,4-triazol-1-ylmethyl)stilbene-4-carbonitrile;
3. (Z)-α-(1,2,4-triazol-1-ylmethyl)-4′-(trifluoromethyl)stilbene-4-carbonitrile;
4. (E)-α-fluoro-α-(1,2,4-triazol-1-ylmethyl)stilbene-4,4′-dicarbonitrile;
5. (Z)-4′-fluoro-α-(imidazol-1-ylmethyl)stilbene-4-carbonitrile;
6. (Z)-2′,4′-dichloro-α-(imidazol-1-ylmethyl)stilbene-4-carbonitrile;
7. (Z)-4′-chloro-α-(imidazol-1-ylmethyl)stilbene-4-carbonitrile;
8. (Z)-α-(imidazol-1-ylmethyl)stilbene-4,4′-dicarbonitrile;
9. (Z)-α-(5-methylimidazol-1-ylmethyl)stilbene-4,4′-dicarbonitrile; and
10. (Z)-2-[2-(4-cyanophenyl)-3-(1,2,4-triazol-1-yl)propenyl]pyridine-5-carbonitrile.

(w) The compounds of formula I as defined in EP-A-299684, especially
1. 2-(4-chlorobenzyl)-2-fluoro-1,3-di(1,2,4-triazol-1-yl)propane;
2. 2-fluoro-2-(2-fluoro-4-chlorobenzyl)-1,3-di(1,2,4-triazol-1-yl)propane;
3. 2-fluoro-2-(2-fluoro-4-trifluoromethylbenzyl)-1,3-di(1,2,4-triazol-1-yl)propane;
4. 3-(4-chlorophenyl)-1-(1,2,4-triazol-1-yl)-2-(1,2,4-triazol-1-ylmethyl)butan-2-ol;
5. 2-(4-chloro-α-fluorobenzyl)-1,3-di(1,2,4-triazol-1-yl)propan-2-ol;
6. 2-(4-chlorobenzyl)-1,3-bis(1,2,4-triazol-1-yl)propane;
7. 4-[2-(4-chlorophenyl)-1,3-di(1,2,4-triazol-1-ylmethyl)ethoxymethyl]-benzonitrile;
8. 1-(4-fluorobenzyl)-2-(2-fluoro-4-trifluoromethylphenyl)-1,3-di(1,2,4-triazol-1-yl)propan-2-ol;
9. 2-(4-chlorophenyl)-1-(4-fluorophenoxy)-1,3-di(1,2,4-triazol-1-yl)propan-2-ol;
10. 1-(4-cyanobenzyl)-2-(2,4-difluorophenyl)-1,3-di(1,2,4-triazol-1-yl)propan-2-ol; and
11. 2-(4-chlorophenyl)-1-phenyl-1,3-di(1,2,4-triazol-1-yl)propan-2-ol.

(x) The compounds as defined in claim 1 of EP A 316097, especially
1. 1,1-dimethyl-8-(1H-1,2,4-triazol-1-ylmethyl)-2(1H)-naphtho[2,1-b]furanone;
2. 1,2-dihydro-1,1-dimethyl-2-oxo-8-(1H-1,2,4-triazol-1-ylmethyl)naphtho[2,1-b]furan-7-carbonitrile;
3. 1,2-dihydro-1,1-dimethyl-2-oxo-8-(1H-1,2,4-triazol-1-ylmethyl)naphtho[2,1-b]furan-7-carboxamide; and
4. 1,2-dihydro-1,1-dimethyl-2-oxo-8-[di(1H-1,2,4-triazol-1-yl)methyl]naphtho[2,1-b]furan-7-carbonitrile.

(y) The compounds of formula (I), as defined in EP A 354689, especially
1. 4-[2-(4-cyanophenyl)-3-(1,2,4-triazol-1-yl)propyl]benzonitrile;
2. 4-[1-(4-chlorobenzyl)-2-(1,2,4-triazol-1-yl)ethyl]benzonitrile;
3. 4-[2-(1,2,4-triazol-1-yl)-1-(4-[trifluoromethyl]benzyl)ethyl]benzonitrile; and
4. 4-[2-(1,2,4-triazol-1-yl)-1-(4-[trifluoromethoxy]benzyl)ethyl]benzonitrile.

(z) The compounds of formula (1), as defined in EP A 354683, especially
1. 6-[2-(4-cyanophenyl)-3-(1,2,4-triazol-1-yl)-propyl]nicotinonitrile; and
2. 4-[1-(1,2,4-triazol-1-yl-methyl)-2-(5-[trifluoromethyl]pyrid-2-yl)ethyl]benzonitrile.

Examples of steroidal aromatase inhibitors that may be mentioned are:

(aa) The compounds of formula (I), as defined in EP A 181287. These are especially the compounds of formula (I) 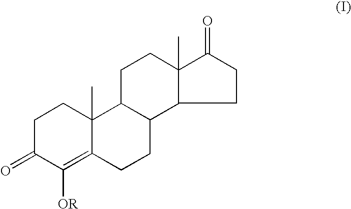
wherein R is hydrogen, acetyl, heptanoyl or benzoyl.

A preferred compound of this group is 4-hydroxy-4-androstene-3,17-dione.

(ab) The compounds as defined in the claims of U.S. Pat. No. 4,322,416, especially 10-(2-propynyl)-oestr-4-ene-3,17-dione.

(ac) The compounds as defined in the claims of DE A 3622841, especially 6-methyleneandrosta-1,4-diene-3,17-dione.

(ad) The compounds as defined in the claims of GB A 2171100, especially 4-aminoandrosta-1,4,6-triene-3,17-dione.

(ae) androsta-1,4,6-triene-3,17-dione.

The content of the patent applications mentioned above under (a)-(z) and (aa)-(ad), especially the subgroups of compounds disclosed therein and the individual compounds disclosed therein as examples, as well as the description of the synthesis and the stated pharmaceutical preparations of these compounds, are incorporated herein by reference.

The general terms used to define the aromatase inhibitors mentioned above under (a)-(r) have the following meanings:

Organic radicals designated by the term “lower” contain up to and including 7 carbon atoms, preferably up to and including 4 carbon atoms.

Acyl is especially lower alkanoyl.

Aryl is, e.g., phenyl or 1- or 2-naphthyl, each of which is unsubstituted or substituted by lower alkyl, hydroxy, lower alkoxy, lower alkanoyloxy, amino, lower alkylamino, di-lower alkylamino, lower alkanoylamino or by halogen.

Any reference hereinbefore and hereinafter to a free bisphosphonate or a free aromatase inhibitor is to be understood as referring also to the corresponding pharmaceutically acceptable salts thereof, as appropriate and expedient.

The aromatase inhibitors can also be used in the form of their hydrates or include other solvents used for their crystallisation.

The most preferred aromatase inhibitor for use in the invention is 4-[α-(4-cyanophenyl)-1-(1,2,4-triazolyl)methyl]-benzonitrile (letrozole) or a pharmaceutically acceptable salt thereof.

Letrozole can be prepared as described in U.S. Pat. No. 5,473,078 (the '078 patent). It can be administered, e.g., as described in U.S. Pat. No. 4,978,672 or the '078 patent, or in the form as it is marketed, e.g., under the trademark FEMARA™ or FEMAR™.

Estrogen receptor antagonists, also referred to as anti-estrogens, relates to a compound which antagonizes the effect of estrogens at the estrogen receptor level. The term includes, but is not limited to tamoxifen, fulvestrant, raloxifene and raloxifene hydrochloride. Tamoxifen can be administered in the form as it is marketed, e.g., NOLVADEX; and raloxifene hydrochloride is marketed as EVISTA. Fulvestrant can be formulated as disclosed in U.S. Pat. No. 4,659,516 and is marketed as FASLODEX.

The Agents of the Invention, i.e., the dual EGFR and VEGF protein tyrosine kinase inhibitor and either the aromatase inhibitor or inhibitor of estrogen action, are preferably used in the form of pharmaceutical preparations that contain the relevant therapeutically effective amount of each active ingredient (either separately or in combination) optionally together with or in admixture with inorganic or organic, solid or liquid, pharmaceutically acceptable carriers which are suitable for administration. The Agents of the Invention may be present in the same pharmaceutical compositions, though are preferably in separate pharmaceutical compositions. Thus the active ingredients may be administered at the same time, e.g., simultaneously, or at different times, e.g., sequentially, and over different periods of time, which may be separate from one another or overlapping.

The pharmaceutical compositions may be, e.g., compositions for enteral, such as oral, rectal, aerosol inhalation or nasal administration; compositions for parenteral, such as intravenous or subcutaneous administration, or compositions for transdermal administration, e.g., passive or iontophoretic.

The particular mode of administration and the dosage may be selected by the attending physician taking into account the particulars of the patient, especially age, weight, life style, activity level, and disease state as appropriate, as well as the particular protein tyrosine kinase inhibitor and either the particular aromatase inhibitor chosen or the particular estrogen receptor antagonist chosen.

The dual EGFR and VEGF protein tyrosine kinase inhibitor may be adapted to oral administration.

The dosage of the dual EGFR and VEGF protein tyrosine kinase inhibitor for use in the invention may depend of various factors, such as effectiveness and duration of action of the active ingredient, mode of administration, sex, age, weight and individual condition of the patient.

The dual EGFR and VEGF protein tyrosine kinase inhibitor formulations comprise from approximately 1% to approximately 95% active ingredient, single-dose administration forms comprising in the preferred embodiment from approximately 20% to approximately 90% active ingredient and forms that are not of single-dose type comprising in the preferred embodiment from approximately 5% to approximately 20% active ingredient. Unit dose forms are, e.g., coated and uncoated tablets, ampoules, vials, suppositories or capsules. Examples are capsules containing from about 0.05 g to about 1.0 g of active substance.

The dual EGFR and VEGF protein tyrosine kinase inhibitor formulations can be administered as such or in the form of pharmaceutical compositions, prophylactically or therapeutically, preferably in an amount effective against the said diseases, to a warm-blooded animal, e.g., a human, requiring such treatment, the compounds especially being used in the form of pharmaceutical compositions. In the case of an individual having a bodyweight of about 70 kg the daily dose administered is from approximately 0.1 g to approximately 5 g, preferably from approximately 0.2 g to approximately 1.5 g, of a compound of the present invention.

Preferably, the dual EGFR and VEGF protein tyrosine kinase inhibitor of the present invention is administered in doses which are in the same order of magnitude as those used in the treatment of the diseases classically treated with dual EGFR and VEGF protein tyrosine kinase inhibitors, such as non small-cell lung cancers, squameous carcinoma (head and neck), breast, gastric, ovarian, colon and prostate cancers and gliomas. In other words, preferably the dual EGFR and VEGF protein tyrosine kinase inhibitor of the present invention is administered in doses which would likewise be therapeutically effective in the treatment of non-small cell lung cancers, squameous carcinoma (head and neck), breast, gastric, ovarian, colon and prostate cancers and gliomas. For example, for the preferred dual EGFR and VEGF protein tyrosine kinase inhibitor, e.g., {6-[4-(4-ethyl-piperazin-1-ylmethyl)-phenyl]-7H-pyrrolo[2,3-d]pyrimidin-4-yl)-(1-[phenyl-ethyl)-amine and salts thereof, doses are in the range from approximately 0.1 g to approximately 5 g, preferably from approximately 0.5 g to approximately 2 g, may be used for treatment of human patients.

Preferably, the aromatase pharmaceutical compositions are adapted for oral or parenteral (especially oral) administration. Intravenous and oral, first and foremost oral, administration is considered to be of particular importance. Preferably the aromatase inhibitor active ingredient is in oral form.

Similarly the dosage of aromatase inhibitor administered is dependent on the species of warm-blooded animal (mammal), the body weight, age and individual condition, and on the form of administration. The applied dosage of the aromatase inhibitor may range between about 0.001 and 30.0 mg/kg, preferably between about 0.001-5 mg/kg.

Aromatase inhibitor formulations in single dose unit form contain preferably from about 1% to about 90%, and formulations not in single dose unit form contain preferably from about 0.1% to about 20%, of the active ingredient. Single dose unit forms, such as capsules, tablets or dragées contain, e.g., from about 1 mg to about 100 mg of the aromatase inhibitor.

Letrozole is preferably administered daily according to the package insert at a dose of 2.5 mg.

In accordance with the present invention, therapy with an estrogen antagonist refers to the standard treatment regimen with such agents for a period of time that such agents are expected to remain effective in preventing disease recurrence and/or death. Generally, therapy with an estrogen antagonist continues for up to about 6 years, e.g., from about 6 months to about 6 years, preferably about 4.5 years to about 6 years, optimally about 5 years. Accordingly, therapy with tamoxifen generally refers to administration of 20-40 mg of tamoxifen daily (30.4-60.8 mg of tamoxifen citrate daily) for a period of up to 6 years.

Pharmaceutical preparations comprising Agents of the Invention for enteral and parenteral administration are, e.g., those in dosage unit forms, such as dragées, tablets or capsules and also ampoules. They are prepared in a manner known per se, e.g., by means of conventional mixing, granulating, confectioning, dissolving or lyophilizing processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, e.g., in admixture with fillers, such as lactose; binders, such as starches; and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intravenously, intramuscularly, intraperitoneally, intranasally, intradermally or subcutaneously. Such fluids are preferably isotonic aqueous solutions or suspensions which can be prepared before use, e.g., from lyophilised preparations which contain the active ingredient alone or together with a pharmaceutically acceptable carrier. The pharmaceutical preparations may be sterilised and/or contain adjuncts, e.g., preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmaceutically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

When the combination partners of the present invention are applied in the form as marketed as single drugs, their dosage and mode of administration can take place in accordance with the information provided on the package insert of the respective marketed drug in order to result in the beneficial effect described herein, if not mentioned herein otherwise.

The following examples are intended to illustrate the invention and are not to be construed as being limitations thereon.

### EXAMPLE 1

### Inhibition of the Dual EGFR and VEGF Protein Tyrosine Kinase Inhibitor, and Either an Aromatase Inhibitor or an Estrogen Receptor Antagonist

The effect of 6-[4-(4-ethyl-piperazin-1-ylmethyl)-phenyl]-7H-pyrrolo[2,3-d]pyrimidin-4-yl}-(1-[phenyl-ethyl)-amine in combination with either letrozole or tamoxifen on cell proliferation, ER-□-mediated transcription and cell cycle in vitro were examined together with the effects of 6-[4-(4-ethyl-piperazin-1-ylmethyl)-phenyl]-7H-pyrrolo[2,3-d]pyrimidin-4-yl}-(1-[phenyl-ethyl)-amine on phosphorylated HER2, MAPK (ERK1/ERK2) and AKT.

Human breast cancer cell lines with varying expression of ER and HER2 were engineered to express aromatase. The cell lines were MCF7 (ER+,HER2−), ZR75.1 (ER+,HER2+), BT474 (ER+, HER2++) and SKBR3 (ER−, HER2++). Clones exhibiting aromatase activity of 2-12 pmoles/mg protein per hour were selected.

Proliferation assays were carried out as follows. The target cell lines listed above were cultured in phenol red-free RPMI 1640 medium containing 10% charcoal stripped foetal calf serum and 10□g.ml insulin (DCC medium), with daily changes for 3 days. The cell lines were then seeded in to 12-well plates at an appropriate density (MCF7 1×10⁴/well, ZR75.1 2×10⁴/well, BT474 2×10⁴/well, SKBR3 1×10⁴/well). After 24-48 hours, the cell monolayers were treated with increasing concentrations of androstenedione or E2. Additionally, the inhibitory effect of tamoxifen, letrozole or 6-[4-(4-ethyl-piperazin-1-ylmethyl)-phenyl]-7H-pyrrolo[2,3-d]pyrimidin-4-yl}-(1-[phenyl-ethyl)-amine alone or in combination was assessed. Drug treatments were changed daily over a period of 6 days. Cell number was determined using a Z1 Coulter Counter (Beckman Coulter, UK). With the threshold set at 15 μm diameter, 75% the average 20 μm size of MCF7 cells. Cell counts were averaged for each triplicate treatment and normalised against the control sample to give the average fold-difference in cell growth.

ERα transactivation was monitored using a standard reporter construct consisting of two EREs upstream of a minimal thymidine kinase reporter linked to a gene encoding luciferase (pEREtkIIluc). Activity was normalized using a second plasmid consisting of the β-galactosisdase gene downstream of the constitutively active simian virus 40 promoter (SV40)(pCH110). The target cell lines indicated, were cultured in DCC medium as described previously. Cells were then seeded into 24-well plates at a density of 7×10⁵ cells/well (MCF7, ZR75.1 and SKBR3). The following day the cells were transfected by lipofectin (Life Technologies, Inc.) at a ratio of 1:2.5 (DNA:lipid) with 0.25 μg of EREIItkluc and 0.25 μg of pCH110 for 4 hours in serum-free phenol red-free RPMI 1640 medium. The cells were subsequently placed in DCC medium and left to recover overnight. BT474 cells were treated similarly but with the exception that cells were seeded at a density of 1×10⁵ cells/well and monolayers were transfected by lipofectamine 2000 (Life Technologies, Inc.) at a ratio of 1:2.5 with 0.4 μg pEREtkIIluc and 0.4 μg pCH110 per well for 4 hours. Transiently transfected monolayers were subsequently treated in each case with the appropriate concentration of androstenedione, tamoxifen, letrozole, 6-[4-(4-ethyl-piperazin-1-ylmethyl)-phenyl]-7H-pyrrolo[2,3-d]pyrimidin-4-yl}-(1-[phenyl-ethyl)-amine or a combination of the agents. After treatment for 24 hours, the luciferase (Promega, UK) and β-galactosidase (Galacton Star, PE Biosystems, UK) activity were measured using a luminometer.

Cell lines were depleted of steroid for 3 days in DCC medium then seeded in 10 cm dishes at a density of 1×10⁶ cells. After 48 hours, appropriate monolayers were pretreated with 6-[4-(4-ethyl-piperazin-1-ylmethyl)-phenyl]-7H-pyrrolo[2,3-d]pyrimidin-4-yl}-(1-[phenyl-ethyl)-amine for 1 hour in DCC medium. Monolayers were then challenged with androstenedione (10⁻⁸M) alone or in combination with 4-OH Tamoxifen (10⁻⁷ M), letrozole (10⁻⁷ M), 6-[4-(4-ethyl-piperazin-1-ylmethyl)-phenyl]-7H-pyrrolo[2,3-d]pyrimidin-4-yl}-(1-[phenyl-ethyl)-amine (1 μM) or a combination of these agents. In the case of MCF7 and ZR75.1 the 6-[4-(4-ethyl-piperazin-1-ylmethyl)-phenyl]-7H-pyrrolo[2,3-d]pyrimidin-4-yl}-(1-[phenyl-ethyl)-amine concentration was increased to 5 μM. Cell were treated for 72 hours then fixed with 70% ethanol and stained with Propridium iodide. FACS analysis was used to establish the effect of the drug combinations on cell cycle.

Western Blot analysis of the effect of 6-[4-(4-ethyl-piperazin-1-ylmethyl)-phenyl]-7H-pyrrolo[2,3-d]pyrimidin-4-yl}-(1-[phenyl-ethyl)-amine on HER2 signal transduction.

Target cell lines were stripped of steroid for 3 days in DCC medium prior to seeding at a density of 1.5-2×10⁶ cells per 10 cm dish. Monolayers were left to acclimatize for 24 hours prior to treatment with 0, 0.1, 1 and 10 μM 6-[4-(4-ethyl-piperazin-1-ylmethyl)-phenyl]-7H-pyrrolo[2,3-d]pyrimidin-4-yl}-(1-[phenyl-ethyl)-amine in DCC medium for 8 hours. Cell monolayers were washed with ice-cold PBS then lysed in extraction buffer (1% (v/v) Triton X100, 10 mM Tris-HCl, pH 7.4, 5 mM EDTA, 50 mM NaCl, 50 mM sodium fluoride, 2 mM Na₃VO₄ and 1 tablet of Complete inhibitor mix (Boehringer UK) per 10 mL of buffer) and homogenised by passage through a 26-gauge needle 6 times. The lysate was incubated on ice for 10 minutes then clarified by centrifugation (14,000 rpm for 10 minutes at 4° C.). The protein concentration was then quantified using BioRad protein assay kit (Bio Rad, UK). Equal amounts of protein (50 μg) were resolved by SDS-PAGE and transferred to nitrocellulose filters (Schleicher and Schuell, UK). Filters were blocked (10 mM Tris-HCl, pH 8.3, 150 mM NaCl containing 4% milk powder) washed twice in PBS containing 0.025% Tween-20 and probed with specific antibodies (p HER2 and total HER2 (Upstate, UK), pAKT, total AKT, pMAPK and total MAPK (Cell signaling, UK) and HRP-conjugated secondary antibody (Amersham Pharmacia Biotech, UK). The antibodies were diluted 1/1000 in 1.5% bovine serum albumin, 10 mM Tris-HCl, pH 8.3, 150 mM NaCl, 0.025% Tween-20 and 0.01% sodium azide. Immuno complexes were detected using Ultra-Signal chemiluminescence kit (Pierce and Warriner, UK). Chemiluminescence was quantified using Fluro-S and analysed using Quantity One software (BioRad, UK).

E2 and androstenedione (AND) induced a proliferative response to ER+ cell lines and increased ER-□-mediated transcription in a dose-dependent manner. Proliferation and transcription were inhibited by tamoxifen or letrozole in ER+ cells, see table below and FIGS. 1-4:

| | MCF7 | ZR75.1 | BT474 |
|---|---|---|---|
| Compound | IC₅₀ [nM] | IC₅₀ [μM] | IC₅₀ [μM] |
| Tamoxifen | 10 | 10 | 10 |
| Letrozole | 5 | 1 | 5 |

FIG. 1 shows that clones from the cell lines MCF7, ZR75.1 and BT474 have a dose dependent increase in proliferation response to E2. Data is expressed as fold change compared to DCC medium alone. The data represented is an average of triplicate readings. Bars show sem. The data was confirmed in two independent experiments.

FIG. 2 shows that clones from the cell lines MCF7, ZR75.1 and BT474 show a dose dependent increase in proliferation in response to increasing doses of androstenedione, whilst clones expressing the vector backbone neo are unresponsive. Data is expressed as fold change compared to DCC medium alone. The data represented is an average of triplicate readings. Bars show sem. The data was confirmed in two independent experiments.

FIG. 3 shows that clones from the cell lines MCF7, ZR75.1 and BT474 show a dose dependent decrease in proliferation in response to increasing doses of 4-OH tamoxifen. Data is expressed as fold change compared to androstenedione alone. The data represented is an average of triplicate readings. Bars show sem. The data was confirmed in a minimum of three independent experiments.

FIG. 4 shows that clones from the cell lines MCF7, ZR75.1 and BT474 show a dose dependent decrease in proliferation in response to increasing doses of letrozole whilst clones expressing the backbone vector neo remain unresponsive. Data is expressed as fold change compared to androstenedione alone. The data represented is an average of triplicate readings. Bars show sem. The data was confirmed in a minimum of three independent experiments.

The effect of 6-[4-(4-ethyl-piperazin-1-ylmethyl)-phenyl]-7H-pyrrolo[2,3-d]pyrimidin-4-yl}-(1-[phenyl-ethyl)-amine on proliferation varied between cell lines according to their EGRF/HER2 status, see table below and FIG. 5.

| | MCF7 | ZR75.1 | SKBR3 | BT474 |
|---|---|---|---|---|
| Compound | IC₅₀ [μM] | IC₅₀ [μM] | IC₅₀ [μM] | IC₅₀ [μM] |
| 6-[4-(4-ethyl-piperazin- | 5-10 | 2-5 | 1.5 | 0.5 |
| 1-ylmethyl)-phenyl]-7H- | | | | |
| pyrrolo[2,3-d]pyrimidin- | | | | |
| 4-yl}-(1-[phenyl- | | | | |
| ethyl)-amine | | | | |

FIG. 5 shows the sensitivity of the cell lines to 6-[4-(4-ethyl-piperazin-1-ylmethyl)-phenyl]-7H-pyrrolo[2,3-d]pyrimidin-4-yl}-(1-[phenyl-ethyl)-amine appears dependent on their HER2 status. Data is expressed as fold change compared to DCC medium alone. The data represented is an average of triplicate readings. Bars show sem. The data was confirmed in a minimum of three independent experiments.

6-[4-(4-ethyl-piperazin-1-ylmethyl)-phenyl]-7H-pyrrolo[2,3-d]pyrimidin-4-yl}-(1-[phenyl-ethyl)-amine caused a dose dependent decrease in basal and AND-induced ER-α-mediated transcription in the cell line BT474 (ER+, HER2++) but showed little effect on cell lines expressing lower levels of HER2.

Combinations of 6-[4-(4-ethyl-piperazin-1-ylmethyl)-phenyl]-7H-pyrrolo[2,3-d]pyrimidin-4-yl}-(1-[phenyl-ethyl)-amine with either letrozole or tamoxifen enhanced the anti-proliferative effects of these agents and FIGS. 6 and 7.

| Compound | MCF7 | ZR75.1 | BT474 |
|---|---|---|---|
| 6-[4-(4-ethyl-piperazin-1- | Increased | Increased | Increased |
| ylmethyl)-phenyl]-7H-pyrrolo | anti- | anti- | anti- |
| [2,3-d]pyrimidin-4-yl}- | proliferation | proliferation | proliferation |
| (1-[phenyl-ethyl)-amine | by | by | by |
| in combination with either | 20-30% | 20-30% | 60-70% |
| letrozole or tamoxifen | | | |

FIG. 6 shows that cell lines were treated with a standard dose of androstenedione and the appropriate cell based IC₅₀ of 6-[4-(4-ethyl-piperazin-1-ylmethyl)-phenyl]-7H-pyrrolo[2,3-d]pyrimidin-4-yl}-(1-[phenyl-ethyl)-amine combined with increasing doses of 4-OH tamoxifen compared to 4-OH tamoxifen alone. Data is expressed as fold change compared to androstenedione alone. The data represented is an average of triplicate readings. Bars show sem. The data was confirmed in a minimum of three independent experiments.

FIG. 7 shows that cell lines were treated with a standard dose of androstenedione and the appropriate cell based IC₅₀ of 6-[4-(4-ethyl-piperazin-1-ylmethyl)-phenyl]-7H-pyrrolo[2,3-d]pyrimidin-4-yl}-(1-[phenyl-ethyl)-amine combined with increasing doses of letrozole compared to letrozole alone. Data is expressed as fold change compared to androstenedione alone. The data represented is an average of triplicate readings. Bars show sem. The data was confirmed in a minimum of three independent experiments.

Combinations of 6-[4-(4-ethyl-piperazin-1-ylmethyl)-phenyl]-7H-pyrrolo[2,3-d]pyrimidin-4-yl}-(1-[phenyl-ethyl)-amine with either letrozole or tamoxifen had no effect of ER-mediated transcription, in the target cell lines MCF7 and ZR75.1 which express low levels of HER2. However, ER-α-mediated transcription in the cell line BT474 (ER+, HER2++) was markedly decreased in response to 6-[4-(4-ethyl-piperazin-1-ylmethyl)-phenyl]-7H-pyrrolo[2,3-d]pyrimidin-4-yl}-(1-[phenyl-ethyl)-amine in combination with tamoxifen or letrozole compared to either agent alone. which suggests that 6-[4-(4-ethyl-piperazin-1-ylmethyl)-phenyl]-7H-pyrrolo[2,3-d]pyrimidin-4-yl}-(1-[phenyl-ethyl)-amine acts via an additive/synergistic selective mechanism dependent on the pheno/genotype of the target cell line.

Western blot analysis illustrated that treatment of the cell lines with 6-[4-(4-ethyl-piperazin-1-ylmethyl)-phenyl]-7H-pyrrolo[2,3-d]pyrimidin-4-yl}-(1-[phenyl-ethyl)-amine had no obvious effect on total or pHER2 but resulted in a decrease of pMAPK in the ZR75.1, BT474 and SKBR3 cell lines while having no effect on MCF7 cells. Similarly, pAKT decreased in a dose dependent manner in response to 6-[4-(4-ethyl-piperazin-1-ylmethyl)-phenyl]-7H-pyrrolo[2,3-d]pyrimidin-4-yl}-(1-[phenyl-ethyl)-amine in all of the cell lines tested as shown in FIG. 8.

The effect of 6-[4-(4-ethyl-piperazin-1-ylmethyl)-phenyl]-7H-pyrrolo[2,3-d]pyrimidin-4-yl}-(1-[phenyl-ethyl)-amine on cell cycle progression was examined using ZR75A3, MCF7A2 and BT474A3 cells in combination with tamoxifen or letrozole.

Using propridium iodide staining and the MCF7A2 cell line, tamoxifen, letrozole and 6-[4-(4-ethyl-piperazin-1-ylmethyl)-phenyl]-7H-pyrrolo[2,3-d]pyrimidin-4-yl}-(1-[phenyl-ethyl)-amine resulted in an increase in the number of cells in G0/G1. Most notably, the combination of 6-[4-(4-ethyl-piperazin-1-ylmethyl)-phenyl]-7H-pyrrolo[2,3-d]pyrimidin-4-yl}-(1-[phenyl-ethyl)-amine with tamoxifen or letrozole resulted in a marked elevation in the number of cells in G0/G1 compared to either agent alone. Although less apparent the same profile was evident in the ZR75.1A3 cell line. Analysis of BT474 A3 revealed that letrozole was more effective than tamoxifen at arresting cells in G0/G1 as was 6-[4-(4-ethyl-piperazin-1-ylmethyl)-phenyl]-7H-pyrrolo[2,3-d]pyrimidin-4-yl}-(1-[phenyl-ethyl)-amine. Similarly it was evident that the combination of 6-[4-(4-ethyl-piperazin-1-ylmethyl)-phenyl]-7H-pyrrolo[2,3-d]pyrimidin-4-yl}-(1-[phenyl-ethyl)-amine with tamoxifen or letrozole resulted in a greater number of cells in G0/G1 and a decrease in those in S/G2/M phases compared to either agent alone as described in FIGS. 9-11.

FIG. 9 shows the effect of 4-OH tamoxifen or letrozole±AEE788 on cell cycle arrest in MCF72A cells.

FIG. 10 shows the effect of 4-OH tamoxifen or letrozole±AEE788 on cell cycle arrest in ZR75.1 A3 cells.

FIG. 11 shows the effect of 4-OH tamoxifen or letrozole±AEE788 on cell cycle arrest in BT474 A3 cells.

This data provides evidence that combinations of 6-[4-(4-ethyl-piperazin-1-ylmethyl)-phenyl]-7H-pyrrolo[2,3-d]pyrimidin-4-yl}-(1-[phenyl-ethyl)-amine in breast cancer over expressing EGFR and/or HER2 with letrozole or tamoxifen provide superior anti-tumor activity as compared with single agents.

### EXAMPLE 2

### The Effect of 6-[4-(4-Ethyl-piperazin-1-ylmethyl)-phenyl]-7H-pyrrolo[2,3-d]pyrimidin-4-yl}-(1-[phenyl-ethyl)-amine in Combination with Either Letrozole or Tamoxifen on the Growth of ZR75.1 Tumour Xenografts In Vivo

Experimental study groups consisted of 6 animals. All tumours were grown to approximately 7 mm diameter under E2 support. Once the tumours were established, the E2 pellet was removed and replaced with androstenedione. Mice were then randomised into cages and treated as described below. Tumours were assessed twice weekly by bi-dimensional caliper measurements and volume calculated based on the volume of a sphere 4/3 μr³. Data was expressed as fold change in tumour volume compared to day zero treatment for each treatment group.

Effect of 6-[4-(4-ethyl-piperazin-1-ylmethyl)-phenyl]-7H-pyrrolo[2,3-d]pyrimidin-4-yl}-(1-[phenyl-ethyl)-amine in combination with endocrine therapy was investigated in ZR75.1A3.

Groups:
(i) Vehicle (10% N-methyl-pyrollidone (NMP)/90% polyethylene glycol (PEG 300)). 300 μL p.o. daily.
(ii) +6-[4-(4-ethyl-piperazin-1-ylmethyl)-phenyl]-7H-pyrrolo[2,3-d]pyrimidin-4-yl}-(1-[phenyl-ethyl)-amine (6.7 mg/mL in 10% NMP/90% PEG 300) 150 μL p.o. equivalent to 1 mg dose 3 times a week
(iii) +letrozole (0.17 mg/mL in 10% NMP/90% PEG300) 150 mL p.o. equivalent to 25 μg dose daily.
(iv) +letrozole, +6-[4-(4-ethyl-piperazin-1-ylmethyl)-phenyl]-7H-pyrrolo[2,3-d]pyrimidin-4-yl}-(1-[phenyl-ethyl)-amine (mice received letrozole plus vehicle or 6-[4-(4-ethyl-piperazin-1-ylmethyl)-phenyl]-7H-pyrrolo[2,3-d]pyrimidin-4-yl}-(1-[phenyl-ethyl)-amine daily)
(v) +tamoxifen (3.3 mg/mL in 10% NMP/90% PEG300) 150 mL p.o. equivalent to 500 μg dose daily.
(vi) +tamoxifen+6-[4-(4-ethyl-piperazin-1-ylmethyl)-phenyl]-7H-pyrrolo[2,3-d]pyrimidin-4-yl}-(1-[phenyl-ethyl)-amine (mice received tamoxifen plus vehicle or 6-[4-(4-ethyl-piperazin-1-ylmethyl)-phenyl]-7H-pyrrolo[2,3-d]pyrimidin-4-yl}-(1-[phenyl-ethyl)-amine daily).

ZR75.1 tumour xenografts were markedly inhibited by letrozole as compared to the effects observed with tamoxifen. Use of 6-[4-(4-ethyl-piperazin-1-ylmethyl)-phenyl]-7H-pyrrolo[2,3-d]pyrimidin-4-yl}-(1-[phenyl-ethyl)-amine alone only had a modest inhibitory effect on tumour growth in the magnitude of that seen with tamoxifen. Tamoxifen combined with 6-[4-(4-ethyl-piperazin-1-ylmethyl)-phenyl]-7H-pyrrolo[2,3-d]pyrimidin-4-yl)-(1-[phenyl-ethyl)-amine showed no added benefit as compared with either agent alone. In contrast however, 6-[4-(4-ethyl-piperazin-1-ylmethyl)-phenyl]-7H-pyrrolo[2,3-d]pyrimidin-4-yl}-(1-[phenyl-ethyl)-amine combined with letrozole resulted in a significant decrease in tumour volume as compared with letrozole administered alone as described in FIG. 12.

FIG. 12 shows 4-OH tamoxifen as affective as AEE788. Most noteworthy letrozole appeared more effective than 4-OH tamoxifen alone or in combination with AEE788. The most effective treatment resulting in a marked decrease in tumour volume was AEE788 in combination with letrozole.

1. A combination which comprises:
(a) a dual EGFR and VEGF protein tyrosine kinase inhibitor of formula (I) 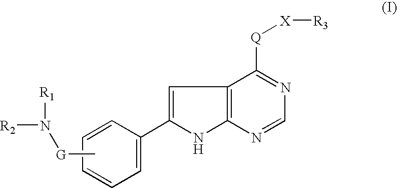
wherein
R₁ and R₂ are, each independently of the other hydrogen, unsubstituted or substituted alkyl or cycloalkyl, a heterocyclic radical bonded via a ring carbon atom, or a radical of the formula R₄—Y—(C=Z)-,
wherein
R₄ is unsubstituted, mono- or disubstituted amino or a heterocyclic radical;
Y is either not present or lower alkyl; and
Z is oxygen, sulfur or imino, with the proviso that R₁ and R₂ are not both hydrogen, or
R₁ and R₂, together with the nitrogen atom to which they are attached, form a heterocyclic radical;
R₃ is a heterocyclic radical organ unsubstituted or substituted aromatic radical;
G is C₁-C₇alkylene, —C(═O)— or C₁-C₆alkylene-C(═O)—, wherein the carbonyl group is attached to the NR₁R₂ moiety;
Q is —NH— or —O—, with the proviso that Q is —O— if G is —C(═O)— or C₁-C₆-alkylene-C(═O)—; and
X is either not present or C₁-C₇-alkylene, with the proviso that a heterocyclic radical R₃ is bonded via a ring carbon atom if X is not present;
or a pharmaceutically acceptable salt thereof or any hydrate thereof; and either
(b) an aromatase inhibitor; or
(c) an estrogen receptor antagonist in which the active ingredients (a) and either (b) or
(c) are present in each case in free form or in the form of a pharmaceutically acceptable salt,
for simultaneous, concurrent, separate or sequential use in the treatment of a disease or condition which responds to dual EGFR and VEGF protein tyrosine kinase inhibition and either aromatase inhibition or inhibition of estrogen action.

2. A method of treating a patient suffering from a disease or condition which responds to dual EGFR and VEGF protein tyrosine kinase inhibition and either aromatase inhibition or inhibition of estrogen action comprising administering to the patient an effective amount of a dual EGFR and VEGF protein tyrosine kinase inhibitor of formula (I) 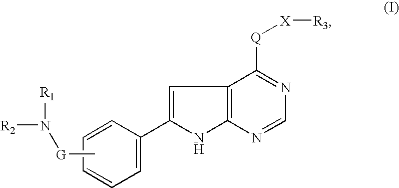
wherein
R₁ and R₂ are, each independently of the other hydrogen, unsubstituted or substituted alkyl or cycloalkyl, a heterocyclic radical bonded via a ring carbon atom, or a radical of the formula R₄—Y—(C=Z)-,
wherein
R₄ is unsubstituted, mono- or disubstituted amino or a heterocyclic radical;
Y is either not present or lower alkyl; and
Z is oxygen, sulfur or imino, with the proviso that R₁ and R₂ are not both hydrogen, or
R₁ and R₂, together with the nitrogen atom to which they are attached, form a heterocyclic radical;
R₃ is a heterocyclic radical or an unsubstituted or substituted aromatic radical;
G is C₁-C₇alkylene, —C(═O)— or C₁-C₆alkylene-C(═O)—, wherein the carbonyl group is attached to the NR₁R₂ moiety;
Q is —NH— or —O—, with the proviso that Q is —O— if G is —C(═O)— or C₁-C₆-alkylene-C(═O)—; and
X is either not present or C₁-C₇-alkylene, with the proviso that a heterocyclic radical R₃ is bonded via a ring carbon atom if X is not present;
or a pharmaceutically acceptable salt thereof or any hydrate thereof and an effective amount of either an aromatase inhibitor or estrogen receptor antagonist.

3. The method of claim 2, for the treatment of a proliferative disease.

4. A package comprising a dual EGFR and VEGF protein tyrosine kinase inhibitor of formula (I) 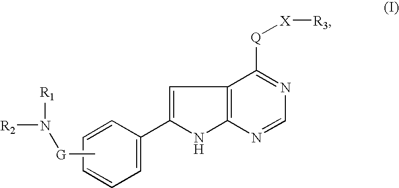
wherein
R₁ and R₂ are, each independently of the other hydrogen, unsubstituted or substituted alkyl or cycloalkyl, a heterocyclic radical bonded via a ring carbon atom, or a radical of the formula R₄—Y—(C=Z)-,
wherein
R₄ is unsubstituted, mono- or disubstituted amino or a heterocyclic radical;
Y is either not present or lower alkyl; and
Z is oxygen, sulfur or imino, with the proviso that R₁ and R₂ are not both hydrogen, or
R₁ and R₂, together with the nitrogen atom to which they are attached, form a heterocyclic radical;
R₃ is a heterocyclic radical or an unsubstituted or substituted aromatic radical;
G is C₁-C₇alkylene, —C(═O)— or C₁-C₆alkylene-C(═O)—, wherein the carbonyl group is attached to the NR₁R₂ moiety;
Q is —NH— or —O—, with the proviso that Q is —O— if G is —C(═O)— or C₁-C₆-alkylene-C(═O)—; and
X is either not present or C₁-C₇-alkylene, with the proviso that a heterocyclic radical R₃ is bonded via a ring-carbon atom if X is not present;
or a pharmaceutically acceptable salt thereof or any hydrate thereof together with instructions for use in combination with either an aromatase inhibitor or estrogen receptor antagonist for treatment of a disease or condition which responds to dual EGFR and VEGF protein tyrosine kinase inhibition and either aromatase inhibition or inhibition of estrogen action.

5. The combination according to claim 1, in which the dual EGFR and VEGF protein tyrosine kinase inhibitor is 6-[4-(4-ethyl-piperazin-1-ylmethyl)-phenyl]-7H-pyrrolo[2,3-d]pyrimidin-4-yl}-(1-[phenyl-ethyl)-amine or a pharmaceutically acceptable salt thereof.

6. The combination according to claim 1, in which the aromatase inhibitor is selected from exemestane, formestane, aminoglutethimide, vorozole, fadrozole, anastrozole, letrozole, roglethimide, pyridoglutethimide, trilostane, testolactone, atamestane, 1-methyl-1,4-androstadiene-3,17-dione, ketokonazole, 4-[α-(4-cyanophenyl)-α-fluoro-1-(1,2,4-triazolyl)methyl]-benzonitrile, 4-[α-(4-cyanophenyl)-α-fluoro-1-(1,2,3-triazolyl)methyl]-benzonitrile and pharmaceutically acceptable salts of these compounds.

7. The combination according to claim 1, in which the aromatase inhibitor is selected from exemestane, formestane, aminoglutethimide, fadrozole, anastrozole, letrozole and pharmaceutically acceptable salts of these compounds.

8. The combination according to claim 1, in which the aromatase inhibitor is a compound of formula (I) 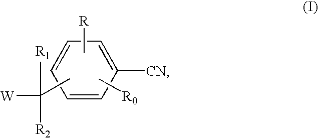
wherein
R and R₀, independently of one another, are each hydrogen or lower alkyl, or
R and R₀ at adjacent carbon atoms, together with the benzene ring to which they are bonded, form a naphthalene or tetrahydronaphthalene ring;
R₁ is hydrogen, lower alkyl, aryl, aryl-lower alkyl or lower alkenyl;
R₂ is hydrogen, lower alkyl, aryl, aryl-lower alkyl, (lower alkyl, aryl or aryl-lower alkyl)-thio or lower alkenyl, or
R₁ and R₂, together, are lower alkylidene or C₄-C₆alkylene;
W is 1-imidazolyl, 1-(1,2,4 or 1,3,4)-triazolyl, 3-pyridyl or one of the mentioned heterocyclic radicals substituted by lower alkyl and aryl within the context of the above definitions has the following meanings: phenyl that is unsubstituted or substituted by one or two substituents from the group lower alkyl, lower alkoxy, hydroxy, lower alkanoyloxy, nitro, amino, halogen, trifluoromethyl, cyano, carboxy, lower alkoxycarbonyl, carbamoyl, N-lower alkylcarbamoyl, N,N-di-lower alkylcarbamoyl, lower alkanoyl, benzoyl, lower alkylsulfonyl, sulfamoyl, N-lower alkylsulfamoyl and N,N-di-lower alkylsulfamoyl; also thienyl, indolyl, pyridyl or furyl, or one of the four last-mentioned heterocyclic radicals monosubstituted by lower alkyl, lower alkoxy, cyano or by halogen;
or a pharmaceutically acceptable salt thereof.

9. The combination according to claim 1, in which the aromatase inhibitor is 4-[α-(4-cyanophenyl)-1-(1,2,4-triazolyl)methyl]-benzonitrile (letrozole) or a pharmaceutically acceptable salt thereof.

10. The combination according to claim 1, in which the estrogen receptor antagonist is selected from tamoxifen, fulvestrant, raloxifene, and raloxifene hydrochloride.

11. The combination in which the estrogen receptor is tamoxifen.

12. A method of reducing cell proliferation in hormone receptor positive cells comprising administering to said patient an effective amount of a dual EGFR and VEGF protein tyrosine kinase inhibitor of formula (I) 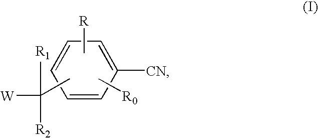
wherein
R and R₀, independently of one another, are each hydrogen or lower alkyl, or
R and R₀ at adjacent carbon atoms, together with the benzene ring to which they are bonded, form a naphthalene or tetrahydronaphthalene ring;
R₁ is hydrogen, lower alkyl, aryl, aryl-lower alkyl or lower alkenyl;
R₂ is hydrogen, lower alkyl, aryl, aryl-lower alkyl, (lower alkyl, aryl or aryl-lower alkyl)-thio or lower alkenyl, or
R₁ and R₂, together, are lower alkylidene or C₄-C₆alkylene;
W is 1-imidazolyl, 1-(1,2,4 or 1,3,4)-triazolyl, 3-pyridyl or one of the mentioned heterocyclic radicals substituted by lower alkyl and aryl within the context of the above definitions has the following meanings: phenyl that is unsubstituted or substituted by one or two substituents from the group lower alkyl, lower alkoxy, hydroxy, lower alkanoyloxy, nitro, amino, halogen, trifluoromethyl, cyano, carboxy, lower alkoxycarbonyl, carbamoyl, N-lower alkylcarbamoyl, N,N-di-lower alkylcarbamoyl, lower alkanoyl, benzoyl, lower alkylsulfonyl, sulfamoyl, N-lower alkylsulfamoyl and N,N-di-lower alkylsulfamoyl; also thienyl, indolyl, pyridyl or furyl, or one of the four last-mentioned heterocyclic radicals monosubstituted by lower alkyl, lower alkoxy, cyano or by halogen;
or a pharmaceutically acceptable salt thereof and an effective amount of either an aromatase inhibitor or an estrogen receptor antagonist.

13. The method according to claim 12, wherein the dual EGFR and VEGF protein tyrosine kinase inhibitor is 6-[4-(4-ethyl-piperazin-1-ylmethyl)-phenyl]-7H-pyrrolo[2,3-d]pyrimidin-4-yl}-(1-[phenyl-ethyl)-amine or a pharmaceutically acceptable salt thereof.

14. The method according to claim 12, wherein the aromatase inhibitor is selected from exemestane, formestane, aminoglutethimide, vorozole, fadrozole, anastrozole, letrozole, roglethimide, pyridoglutethimide, trilostane, testolactone, atamestane, 1-methyl-1,4-androstadiene-3,17-dione, ketokonazole, 4-[α-(4-cyanophenyl)-α-fluoro-1-(1,2,4-triazolyl)methyl]-benzonitrile, 4-[α-(4-cyanophenyl)-α-fluoro-1-(1,2,3-triazolyl)methyl]-benzonitrile and pharmaceutically acceptable salts of these compounds.

15. The method according to claim 14, wherein the aromatase inhibitor is selected from exemestane, formestane, aminoglutethimide, fadrozole, anastrozole, letrozole and pharmaceutically acceptable salts of these compounds.

16. The method according to claim 12, wherein the estrogen receptor antagonist is selected from tamoxifen, fulvestrant, raloxifene and raloxifene hydrochloride.

17. The method according to claim 16, wherein the estrogen receptor antagonist is tamoxifen.
